SEQUENCE LISTING (44 sequences)

SEQ ID NO: 1 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 2 - protein, 793 aa, Homo sapiens
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQVEVMVEEKTTESQEETVVMSLKNGQISSEEPKQEEEREQGSDEISHHEKMEEEDKERAEAERARLEAEERERIKAEQDKKIADERARIEAEEKAAAQERERREAEERERMREEEKRAAEERQRIKEEEKRAAEERQRIKEEEKRAAEERQRIKEEEKRAAEERQRARAEEEEKAKVEEQKRNKQLEEKKHAMQETKIKGEKVEQKIEGKWVNEKKAQEDKLQTAVLKKQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 3 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 4 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 5 - protein, 181 aa, Homo sapiens
MGNIFANLFKGLFGKKEMRILMVGLDAAGKTTILYKLKLGEIVTTIPTIGFNVETVEYKNISFTVWDVGGQDKIRPLWRHYFQNTQGLIFVVDSNDRERVNEAREELMRMLAEDELRDAVLLVFANKQDLPNAMNAAEITDKLGLHSLRHRNWYIQATCATSGDGLYEGLDWLSNQLRNQK

SEQ ID NO: 6 - protein, 592 aa, Homo sapiens
MEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 7 - protein, 180 aa, Homo sapiens
MGLTVSALFSRIFGKKQMRILMVGLDAAGKTTILYKLKLGEIVTTIPTIGFNVETVEYKNICFTVWDVGGQDKIRPLWRHYFQNTQGLIFVVDSNDRERVQESADELQKMLQEDELRDAVLLVFANKQDMPNAMPVSELTDKLGLQHLRSRTWYVQATCATQGTGLYDGLDWLSHELSKR

SEQ ID NO: 8 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 9 - protein, 206 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 10 - protein, 142 aa, Homo sapiens
MATKIDKEACRAAYNLVRDDGSAVIWVTFKYDGSTIVPGEQGAEYQHFIQQCTDDVRLFAFVRFTTGDAMSKRSKFALITWIGENVSGLQRAKTGTDKTLVKEVVQNFAKEFVISDRKELEEDFIKSELKKAGGANYDAQTE

SEQ ID NO: 11 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 12 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 13 - protein, 552 aa, Homo sapiens
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 14 - protein, 138 aa, Homo sapiens
MPNFSGNWKIIRSENFEELLKVLGVNVMLRKIAVAAASKPAVEIKQEGDTFYIKTSTTVRTTEINFKVGEEFEEQTVDGRPCKSLVKWESENKMVCEQKLLKGEGPKTSWTRELTNDGELILTMTADDVVCTRVYVRE

SEQ ID NO: 15 - protein, 89 aa, Homo sapiens
MDGEEKTYGGCEGPDAMYVKLISSDGHEFIVKREHALTSGTIKAMLSGPGQFAENETNEVNFREIPSHVLSKVCMYFTYKVRYTNSSTE

SEQ ID NO: 16 - protein, 50 aa, Homo sapiens
MLIKVKTLTGKEIEIDIEPTDKVERIKERVEEKEGIPPQQQRLIYSGKQM

SEQ ID NO: 17 - protein, 132 aa, Homo sapiens
MVDAFLGTWKLVDSKNFDDYMKSLGVGFATRQVASMTKPTTIIEKNGDILTLKTHSTFKNTEISFKLGVEFDETTADDRKVKSIVTLDGGKLVHLQKWDGQETTLVRELIDGKLILTLTHGTAVCTRTYEKE

SEQ ID NO: 18 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 19 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 20 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 21 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 22 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 23 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 24 - protein, 1134 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 25 - protein, 5890 aa, Homo sapiens
MEKEETTRELLLPNWQGSGSHGLTIAQRDDGVFVQEVTQNSPAARTGVVKEGDQIVGATIYFDNLQSGEVTQLLNTMGHHTVGLKLHRKGDRSPEPGQTWTREVFSSCSSEVVLSGDDEEYQRIYTTKIKPRLKSEDGVEGDLGETQSRTITVTRRVTAYTVDVTGREGAKDIDISSPEFKIKIPRHELTEISNVDVETQSGKTVIRLPSGSGAASPTGSAVDIRAGAISASGPELQGAGHSKLQVTMPGIKVGGSGVNVNAKGLDLGGRGGVQVPAVDISSSLGGRAVEVQGPSLESGDHGKIKFPTMKVPKFGVSTGREGQTPKAGLRVSAPEVSVGHKGGKPGLTIQAPQLEVSVPSANIEGLEGKLKGPQITGPSLEGDLGLKGAKPQGHIGVDASAPQIGGSITGPSVEVQAPDIDVQGPGSKLNVPKMKVPKFSVSGAKGEETGIDVTLPTGEVTVPGVSGDVSLPEIATGGLEGKMKGTKVKTPEMIIQKPKISMQDVDLSLGSPKLKGDIKVSAPGVQGDVKGPQVALKGSRVDIETPNLEGTLTGPRLGSPSGKTGTCRISMSEVDLNVAAPKVKGGVDVTLPRVEGKVKVPEVDVRGPKVDVSAPDVEAHGPEWNLKMPKMKMPTFSTPGAKGEGPDVHMTLPKGDISISGPKVNVEAPDVNLEGLGGKLKGPDVKLPDMSVKTPKISMPDVDLHVKGTKVKGEYDVTVPKLEGELKGPKVDIDAPDVDVHGPDWHLKMPKMKMPKFSVPGFKAEGPEVDVNLPKADVDISGPKIDVTAPDVSIEEPEGKLKGPKFKMPEMNIKVPKISMPDVDLHLKGPNVKGEYDVTMPKVESEIKVPDVELKSAKMDIDVPDVEVQGPDWHLKMPKMKMPKFSMPGFKAEGPEVDVNLPKADVDISGPKVGVEVPDVNIEGPEGKLKGPKFKMPEMNIKAPKISMPDVDLHMKGPKVKGEYDMTVPKLEGDLKGPKVDVSAPDVEMQGPDWNLKMPKIKMPKFSMPSLKGEGPEFDVNLSKANVDISAPKVDTNAPDLSLEGPEGKLKGPKFKMPEMHFRAPKMSLPDVDLDLKGPKMKGNVDISAPKIEGEMQVPDVDIRGPKVDIKAPDVEGQGLDWSLKIPKMKMPKFSMPSLKGEGPEVDVNLPKADVVVSGPKVDIEAPDVSLEGPEGKLKGPKFKMPEMHFKTPKISMPDVDLHLKGPKVKGDVDVSVPKVEGEMKVPDVEIKGPKMDIDAPDVEVQGPDWHLKMPKMKMPKFSMPGFKGEGREVDVNLPKADIDVSGPKVDVEVPDVSLEGPEGKLKGPKFKMPEMHFKAPKISMPDVDLNLKGPKLKGDVDVSLPEVEGEMKVPDVDIKGPKVDISAPDVDVHGPDWHLKMPKVKMPKFSMPGFKGEGPEVDVKLPKADVDVSGPKMDAEVPDVNIEGPDAKLKGPKFKMPEMSIKPQKISIPDVGLHLKGPKMKGDYDVTVPKVEGEIKAPDVDIKGPKVDINAPDVEVHGPDWHLKMPKVKMPKFSMPGFKGEGPEVDMNLPKADLGVSGPKVDIDVPDVNLEAPEGKLKGPKFKMPSMNIQTHKISMPDVGLNLKAPKLKTDVDVSLPKVEGDLKGPEIDVKAPKMDVNVGDIDIEGPEGKLKGPKFKMPEMHFKAPKISMPDVDLHLKGPKVKGDMDVSVPKVEGEMKVPDVDIKGPKVDIDAPDVEVHDPDWHLKMPKMKMPKFSMPGFKAEGPEVDVNLPKADIDVSGPSVDTDAPDLDIEGPEGKLKGSKFKMPKLNIKAPKVSMPDVDLNLKGPKLKGEIDASVPELEGDLRGPQVDVKGPFVEAEVPDVDLECPDAKLKGPKFKMPEMHFKAPKISMPDVDLHLKGPKVKGDADVSVPKLEGDLTGPSVGVEVPDVELECPDAKLKGPKFKMPDMHFKAPKISMPDVDLHLKGPKVKGDVDVSVPKLEGDLTGPSVGVEVPDVELECPDAKLKGPKFKMPEMHFKTPKISMPDVDLHLKGPKVKGDMDVSVPKVEGEMKVPDVDIKGPKMDIDAPDVDVHGPDWHLKMPKMKMPKFSMPGFKAEGPEVDVNLPKADVVVSGPKVDVEVPDVSLEGPEGKLKGPKLKMPEMHFKAPKISMPDVDLHLKGPKVKGDVDVSLPKLEGDLTGPSVDVEVPDVELECPDAKLKGPKFKMPEMHFKTPKISMPDVNLNLKGPKVKGDMDVSVPKVEGEMKVPDVDIRGPKVDIDAPDVDVHGPDWHLKMPKMKMPKFSMPGFKGEGPEVDVNLPKADVDVSGPKVDVEVPDVSLEGPEGKLKGPKFKMPEMHFKTPKISMPDVDFNLKGPKIKGDVDVSAPKLEGELKGPELDVKGPKLDADMPEVAVEGPNGKWKTPKFKMPDMHFKAPKISMPDLDLHLKSPKAKGEVDVDVPKLEGDLKGPHVDVSGPDIDIEGPEGKLKGPKFKMPDMHFKAPNISMPDVDLNLKGPKIKGDVDVSVPEVEGKLEVPDMNIRGPKVDVNAPDVQAPDWHLKMPKMKMPKFSMPGFKAEGPEVDVNLPKADVDISGPKVDIEGPDVNIEGPEGKLKGPKLKMPEMNIKAPKISMPDFDLHLKGPKVKGDVDVSLPKVEGDLKGPEVDIKGPKVDINAPDVGVQGPDWHLKMPKVKMPKFSMPGFKGEGPDGDVKLPKADIDVSGPKVDIEGPDVNIEGPEGKLKGPKFKMPEMNIKAPKISMPDIDLNLKGPKVKGDVDVSLPKVEGDLKGPEVDIKGPKVDIDAPDVDVHGPDWHLKMPKIKMPKISMPGFKGEGPDVDVNLPKADIDVSGPKVDVECPDVNIEGPEGKWKSPKFKMPEMHFKTPKISMPDIDLNLTGPKIKGDVDVTGPKVEGDLKGPEVDLKGPKVDIDVPDVNVQGPDWHLKMPKMKMPKFSMPGFKAEGPEVDVNLPKADVDVSGPKVDVEGPDVNIEGPEGKLKGPKFKMPEMNIKAPKIPMPDFDLHLKGPKVKGDVDISLPKVEGDLKGPEVDIRGPQVDIDVPDVGVQGPDWHLKMPKVKMPKFSMPGFKGEGPDVDVNLPKADLDVSGPKVDIDVPDVNIEGPEGKLKGPKFKMPEMNIKAPKISMPDIDLNLKGPKVKGDMDVSLPKVEGDMKVPDVDIKGPKVDINAPDVDVQGPDWHLKMPKIKMPKISMPGFKGEGPEVDVNLPKADLDVSGPKVDVDVPDVNIEGPDAKLKGPKFKMPEMNIKAPKISMPDLDLNLKGPKMKGEVDVSLANVEGDLKGPALDIKGPKIDVDAPDIDIHGPDAKLKGPKLKMPDMHVNMPKISMPEIDLNLKGSKLKGDVDVSGPKLEGDIKAPSLDIKGPEVDVSGPKLNIEGKSKKSRFKLPKFNFSGSKVQTPEVDVKGKKPDIDITGPKVDINAPDVEVQGKVKGSKFKMPFLSISSPKVSMPDVELNLKSPKVKGDLDIAGPNLEGDFKGPKVDIKAPEVNLNAPDVDVHGPDWNLKMPKMKMPKFSVSGLKAEGPDVAVDLPKGDINIEGPSMNIEGPDLNVEGPEGGLKGPKFKMPDMNIKAPKISMPDIDLNLKGPKVKGDVDISLPKLEGDLKGPEVDIKGPKVDINAPDVDVHGPDWHLKMPKVKMPKFSMPGFKGEGPEVDVTLPKADIDISGPNVDVDVPDVNIEGPDAKLKGPKFKMPEMNIKAPKISMPDFDLNLKGPKMKGDVVVSLPKVEGDLKGPEVDIKGPKVDIDTPDINIEGSEGKFKGPKFKIPEMHLKAPKISMPDIDLNLKGPKVKGDVDVSLPKMEGDLKGPEVDIKGPKVDINAPDVDVQGPDWHLKMPKVKMPKFSMPGFKGEGPDVDVNLPKADLDVSGPKVDIDVPDVNIEGPEGKLKGPKFKMPEMNIKAPKISMPDIDLNLKGPKVKGDMDVSLPKVEGDMQVPDLDIKGPKVDINAPDVDVRGPDWHLKMPKIKMPKISMPGFKGEGPEVDVNLPKADLDVSGPKVDVDVPDVNIEGPDAKLKGPKFKMPEMNIKAPKISMPDFDLHLKGPKVKGDVDVSLPKMEGDLKAPEVDIKGPKVDIDAPDVDVHGPDWHLKMPKVKMPKFSMPGFKGEGPEVDVNLPKADIDVSGPKVDIDTPDIDIHGPEGKLKGPKFKMPDLHLKAPKISMPEVDLNLKGPKMKGDVDVSLPKVEGDLKGPEVDIKGPKVDIDVPDVDVQGPDWHLKMPKVKMPKFSMPGFKGEGPDVDVNLPKADLDVSGPKVDIDVPDVNIEGPDAKLKGPKFKMPEMNIKAPKISMPDFDLHLKGPKVKGDVDVSLPKVEGDLKGPEVDIKGPKVDIDAPDVDVHGPDWHLKMPKVKMPKFSMPGFKGEGPDVDVTLPKADIEISGPKVDIDAPDVSIEGPDAKLKGPKFKMPEMNIKAPKISMPDIDFNLKGPKVKGDVDVSLPKVEGDLKGPEIDIKGPSLDIDTPDVNIEGPEGKLKGPKFKMPEMNIKAPKISMPDFDLHLKGPKVKGDVDVSLPKVESDLKGPEVDIEGPEGKLKGPKFKMPDVHFKSPQISMSDIDLNLKGPKIKGDMDISVPKLEGDLKGPKVDVKGPKVGIDTPDIDIHGPEGKLKGPKFKMPDLHLKAPKISMPEVDLNLKGPKVKGDMDISLPKVEGDLKGPEVDIRDPKVDIDVPDVDVQGPDWHLKMPKVKMPKFSMPGFKGEGPDVDVNLPKADIDVSGPKVDVDVPDVNIEGPDAKLKGPKFKMPEMSIKAPKISMPDIDLNLKGPKVKGDVDVTLPKVEGDLKGPEADIKGPKVDINTPDVDVHGPDWHLKMPKVKMPKFSMPGFKGEGPDVDVSLPKADIDVSGPKVDVDIPDVNIEGPDAKLKGPKFKMPEINIKAPKISIPDVDLDLKGPKVKGDFDVSVPKVEGTLKGPEVDLKGPRLDFEGPDAKLSGPSLKMPSLEISAPKVTAPDVDLHLKAPKIGFSGPKLEGGEVDLKGPKVEAPSLDVHMDSPDINIEGPDVKIPKFKKPKFGFGAKSPKADIKSPSLDVTVPEAELNLETPEISVGGKGKKSKFKMPKIHMSGPKIKAKKQGFDLNVPGGEIDASLKAPDVDVNIAGPDAALKVDVKSPKTKKTMFGKMYFPDVEFDIKSPKFKAEAPLPSPKLEGELQAPDLELSLPAIHVEGLDIKAKAPKVKMPDVDISVPKIEGDLKGPKVQANLGAPDINIEGLDAKVKTPSFGISAPQVSIPDVNVNLKGPKIKGDVPSVGLEGPDVDLQGPEAKIKFPKFSMPKIGIPGVKMEGGGAEVHAQLPSLEGDLRGPDVKLEGPDVSLKGPGVDLPSVNLSMPKVSGPDLDLNLKGPSLKGDLDASVPSMKVHAPGLNLSGVGGKMQVGGDGVKVPGIDATTKLNVGAPDVTLRGPSLQGDLAVSGDIKCPKVSVGAPDLSLEASEGSIKLPKMKLPQFGISTPGSDLHVNAKGPQVSGELKGPGVDVNLKGPRISAPNVDFNLEGPKVKGSLGATGEIKGPTVGGGLPGIGVQGLEGNLQMPGIKSSGCDVNLPGVNVKLPTGQISGPEIKGGLKGSEVGFHGAAPDISVKGPAFNMASPESDFGINLKGPKIKGGADVSGGVSAPDISLGEGHLSVKGSGGEWKGPQVSSALNLDTSKFAGGLHFSGPKVEGGVKGGQIGLQAPGLSVSGPQGHLESGSGKVTFPKMKIPKFTFSGRELVGREMGVDVHFPKAEASIQAGAGDGEWEESEVKLKKSKIKMPKFNFSKPKGKGGVTGSPEASISGSKGDLKSSKASLGSLEGEAEAEASSPKGKFSLFKSKKPRHRSNSFSDEREFSGPSTPTGTLEFEGGEVSLEGGKVKGKHGKLKFGTFGGLGSKSKGHYEVTGSDDETGKLQGSGVSLASKKSRLSSSSSNDSGNKVGIQLPEVELSVSTKKE

SEQ ID NO: 26 - protein, 2468 aa, Homo sapiens
MATVVVEATEPEPSGSIANPAASTSPSLSHRFLDSKFYLLVVVGEIVTEEHLRRAIGNIELGIRSWDTNLIECNLDQELKLFVSRHSARFSPEVPGQKILHHRSDVLETVVLINPSDEAVSTEVRLMITDAARHKLLVLTGQCFENTGELILQSGSFSFQNFIEIFTDQEIGELLSTTHPANKASLTLFCPEEGDWKNSNLDRHNLQDFINIKLNSASILPEMEGLSEFTEYLSESVEVPSPFDILEPPTSGGFLKLSKPCCYIFPGGRGDSALFAVNGFNMLINGGSERKSCFWKLIRHLDRVDSILLTHIGDDNLPGINSMLQRKIAELEEEQSQGSTTNSDWMKNLISPDLGVVFLNVPENLKNPEPNIKMKRSIEEACFTLQYLNKLSMKPEPLFRSVGNTIDPVILFQKMGVGKLEMYVLNPVKSSKEMQYFMQQWTGTNKDKAEFILPNGQEVDLPISYLTSVSSLIVWHPANPAEKIIRVLFPGNSTQYNILEGLEKLKHLDFLKQPLATQKDLTGQVPTPVVKQTKLKQRADSRESLKPAAKPLPSKSVRKESKEETPEVTKVNHVEKPPKVESKEKVMVKKDKPIKTETKPSVTEKEVPSKEEPSPVKAEVAEKQATDVKPKAAKEKTVKKETKVKPEDKKEEKEKPKKEVAKKEDKTPIKKEEKPKKEEVKKEVKKEIKKEEKKEPKKEVKKETPPKEVKKEVKKEEKKEVKKEEKEPKKEIKKLPKDAKKSSTPLSEAKKPAALKPKVPKKEESVKKDSVAAGKPKEKGKIKVIKKEGKAAEAVAAAVGTGATTAAVMAAAGIAAIGPAKELEAERSLMSSPEDLTKDFEELKAEEVDVTKDIKPQLELIEDEEKLKETEPVEAYVIQKEREVTKGPAESPDEGITTTEGEGECEQTPEELEPVEKQGVDDIEKFEDEGAGFEESSETGDYEEKAETEEAEEPEEDGEEHVCVSASKHSPTEDEESAKAEADAYIREKRESVASGDDRAEEDMDEAIEKGEAEQSEEEADEEDKAEDAREEEYEPEKMEAEDYVMAVVDKAAEAGGAEEQYGFLTTPTKQLGAQSPGREPASSIHDETLPGGSESEATASDEENREDQPEEFTATSGYTQSTIEISSEPTPMDEMSTPRDVMSDETNNEETESPSQEFVNITKYESSLYSQEYSKPADVTPLNGFSEGSKTDATDGKDYNASASTISPPSSMEEDKFSRSALRDAYCSEVKASTTLDIKDSISAVSSEKVSPSKSPSLSPSPPSPLEKTPLGERSVNFSLTPNEIKVSAEAEVAPVSPEVTQEVVEEHCASPEDKTLEVVSPSQSVTGSAGHTPYYQSPTDEKSSHLPTEVIEKPPAVPVSFEFSDAKDENERASVSPMDEPVPDSESPIEKVLSPLRSPPLIGSESAYESFLSADDKASGRGAESPFEEKSGKQGSPDQVSPVSEMTSTSLYQDKQEGKSTDFAPIKEDFGQEKKTDDVEAMSSQPALALDERKLGDVSPTQIDVSQFGSFKEDTKMSISEGTVSDKSATPVDEGVAEDTYSHMEGVASVSTASVATSSFPEPTTDDVSPSLHAEVGSPHSTEVDDSLSVSVVQTPTTFQETEMSPSKEECPRPMSISPPDFSPKTAKSRTPVQDHRSEQSSMSIEFGQESPEQSLAMDFSRQSPDHPTVGAGVLHITENGPTEVDYSPSDMQDSSLSHKIPPMEEPSYTQDNDLSELISVSQVEASPSTSSAHTPSQIASPLQEDTLSDVAPPRDMSLYASLTSEKVQSLEGEKLSPKSDISPLTPRESSPLYSPTFSDSTSAVKEKTATCHSSSSPPIDAASAEPYGFRASVLFDTMQHHLALNRDLSTPGLEKDSGGKTPGDFSYAYQKPEETTRSPDEEDYDYESYEKTTRTSDVGGYYYEKIERTTKSPSDSGYSYETIGKTTKTPEDGDYSYEIIEKTTRTPEEGGYSYDISEKTTSPPEVSGYSYEKTERSRRLLDDISNGYDDSEDGGHTLGDPSYSYETTEKITSFPESEGYSYETSTKTTRTPDTSTYCYETAEKITRTPQASTYSYETSDLCYTAEKKSPSEARQDVDLCLVSSCEYKHPKTELSPSFINPNPLEWFASEEPTEESEKPLTQSGGAPPPPGGKQQGRQCDETPPTSVSESAPSQTDSDVPPETEECPSITADANIDSEDESETIPTDKTVTYKHMDPPPAPVQDRSPSPRHPDVSMVDPEALAIEQNLGKALKKDLKEKTKTKKPGTKTKSSSPVKKSDGKSKPLAASPKPAGLKESSDKVSRVASPKKKESVEKAAKPTTTPEVKAARGEEKDKETKNAANASASKSAKTATAGPGTTKTTKSSAVPPGLPVYLDLCYIPNHSNSKNVDVEFFKRVRSSYYVVSGNDPAAEEPSRAVLDALLEGKAQWGSNMQVTLIPTHDSEVMREWYQETHEKQQDLNIMVLASSSTVVMQDESFPACKIEL

SEQ ID NO: 27 - protein, 2511 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

SEQ ID NO: 28 - protein, 286 aa, Homo sapiens
MAEDGEEAEFHFAALYISGQWPRLRADTDLQRLGSSAMAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 29 - protein, 1101 aa, Homo sapiens
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 30 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 31 - protein, 1268 aa, Homo sapiens [sequence omitted]

SEQ ID NO: 32 - protein, 4128 aa, Homo sapiens [sequence omitted]

SEQ ID NO: 33 - protein, 445 aa, Homo sapiens
MNEEYDVIVLGTGLTECILSGIMSVNGKKVLHMDRNPYYGGESASITPLEDLYKRFKIPGSPPESMGRGRDWNVDLIPKFLMANGQLVKMLLYTEVTRYLDFKVTEGSFVYKGGKIYKVPSTEAEALASSLMGLFEKRRFRKFLVYVANFDEKDPRTFEGIDPKKTTMRDVYKKFDLGQDVIDFTGHALALYRTDDYLDQPCYETINRIKLYSESLARYGKSPYLYPLYGLGELPQGFARLSAIYGGTYMLNKPIEEIIVQNGKVIGVKSEGEIARCKQLICDPSYVKDRVEKVGQVIRVICILSHPIKNTNDANSCQIIIPQNQVNRKSDIYVCMISFAHNVAAQGKYIAIVSTTVETKEPEKEIRPALELLEPIEQKFVSISDLLVPKDLGTESQIFISRTYDATTHFETTCDDIKNIYKRMTGSEFDFEEMKRKKNDIYGED

SEQ ID NO: 34 - protein, 529 aa, Homo sapiens
MLGFVGRVAAAPASGALRRLTPSASLPPAQLLLRAAPTAVHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS

SEQ ID NO: 35 - protein, 1045 aa, Homo sapiens
MEGLEEAEANCSVAFAEAQRWVEAVTEKNFETKDFRASLENGVLLCDLINKLKPGVIKKINRLSTPIAGLDNINVFLKACEQIGLKEAQLFHPGDLQDLSNRVTVKQEETDRRVKNVLITLYWLGRKAQSNPYYNGPHLNLKAFENLLGQALTKALEDSSFLKRSGRDSGYGDIWCPERGEFLAPPRHHKREDSFESLDSLGSRSLTSCSSDITLRGGREGFESDTDSEFTFKMQDYNKDDMSYRRISAVEPKTALPFNRFLPNKSRQPSYVPAPLRKKKPDKHEDNRRSWASPVYTEADGTFSRLFQKIYGENGSKSMSDVSAEDVQNLRQLRYEEMQKIKSQLKEQDQKWQDDLAKWKDRRKSYTSDLQKKKEEREEIEKQALEKSKRSSKTFKEMLQDRESQNQKSTVPSRRRMYSFDDVLEEGKRPPTMTVSEASYQSERVEEKGATYPSEIPKEDSTTFAKREDRVTTEIQLPSQSPVEEQSPASLSSLRSRSTQMESTRVSASLPRSYRKTDTVRLTSVVTPRPFGSQTRGISSLPRSYTMDDAWKYNGDVEDIKRTPNNVVSTPAPSPDASQLASSLSSQKEVAATEEDVTRLPSPTSPFSSLSQDQAATSKATLSSTSGLDLMSESGEGEISPQREVSRSQDQFSDMRISINQTPGKSLDFGFTIKWDIPGIFVASVEAGSPAEFSQLQVDDEIIAINNTKFSYNDSKEWEEAMAKAQETGHLVMDVRRYGKAGSPETKWIDATSGIYNSEKSSNLSVTTDFSESLQSSNIESKEINGIHDESNAFESKASESISLKNLKRRSQFFEQGSSDSVVPDLPVPTISAPSRWVWDQEEERKRQERWQKEQDRLLQEKYQREQEKLREEWQRAKQEAERENSKYLDEELMVLSSNSMSLTTREPSLATWEATWSEGSKSSDREGTRAGEEERRQPQEEVVHEDQGKKPQDQLVIERERKWEQQLQEEQEQKRLQAEAEEQKRPAEEQKRQAEIERETSVRIYQYRRPVDSYDIPKTEEASSGFLPGDRNKSRSTTELDDYS

SEQ ID NO: 36 - protein, 759 aa, Homo sapiens
MESSPFNRRQWTSLSLRVTAKELSLVNKNKSSAIVEIFSKYQKAAEETNMEKKRSNTENLSQHFRKGTLTVLKKKWENPGLGAESHTDSLRNSSTEIRHRADHPPAEVTSHAASGAKADQEEQIHPRSRLRSPPEALVQGRYPHIKDGEDLKDHSTESKKMENCLGESRHEVEKSEISENTDASGKIEKYNVPLNRLKMMFEKGEPTQTKILRAQSRSASGRKISENSYSLDDLEIGPGQLSSSTFDSEKNESRRNLELPRLSETSIKDRMAKYQAAVSKQSSSTNYTNELKASGGEIKIHKMEQKENVPPGPEVCITHQEGEKISANENSLAVRSTPAEDDSRDSQVKSEVQQPVHPKPLSPDSRASSLSESSPPKAMKKFQAPARETCVECQKTVYPMERLLANQQVFHISCFRCSYCNNKLSLGTYASLHGRIYCKPHFNQLFKSKGNYDEGFGHRPHKDLWASKNENEEILERPAQLANARETPHSPGVEDAPIAKVGVLAASMEAKASSQQEKEDKPAETKKLRIAWPPPTELGSSGSALEEGIKMSKPKWPPEDEISKPEVPEDVDLDLKKLRRSSSLKERSRPFTVAASFQSTSVKSPKTVSPPIRKGWSMSEQSEESVGGRVAERKQVENAKASKKNGNVGKTTWQNKESKGETGKRSKEGHSLEMENENLVENGADSDEDDNSFLKQQSPQEPKSLNWSSFVDNTFAEEFTTQNQKSQDVELWEGEVVKELSVEEQIKRNRYYDEDEDEE

SEQ ID NO: 37 - protein, 877 aa, Homo sapiens
MLASLYNGSVCVWNHETQTLVKTFEVCDLPVRAAKFVARKNWVVTGADDMQIRVFNYNTLERVHMFEAHSDYIRCIAVHPTQPFILTSSDDMLIKLWDWDKKWSCSQVFEGHTHYVMQIVINPKDNNQFASASLDRTIKVWQLGSSSPNFTLEGHEKGVNCIDYYSGGDKPYLISGADDRLVKIWDYQNKTCVQTLEGHAQNVSCASFHPELPIIITGSEDGTVRIWHSSTYRLESTLNYGMERVWCVASLRGSNNVALGYDEGSIIVKLGREEPAMSMDANGKIIWAKHSEVQQANLKAMGDAEIKDGERLPLAVKDMGSCEIYPQTIQHNPNGRFVVVCGDGEYIIYTAMALRNKSFGSAQEFAWAHDSSEYAIRESNSIVKIFKNFKEKKSFKPDFGAESIYGGFLLGVRSVNGLAFYDWDNTELIRRIEIQPKHIFWSDSGELVCIATEESFFILKYLSEKVLAAQETHEGVTEDGIEDAFEVLGEIQEIVKTGLWVGDCFIYTSSVNRLNYYVGGEIVTIAHLDRTMYLLGYIPKDNRLYLGDKELNIISYSLLVSVLEYQTAVMRRDFSMADKVLPTIPKEQRTRVAHFLEKQGFKQQALTVSTDPEHRFELALQLGELKIAYQLAVEAESEQKWKQLAELAISKCQFGLAQECLHHAQDYGGLLLLATASGNANMVNKLAEGAERDGKNNVAFMSYFLQGKVDACLELLIRTGRLPEAAFLARTYLPSQVSRVVKLWRENLSKVNQKAAESLADPTEYENLFPGLKEAFVVEEWVKETHADLWPAKQYPLVTPNEERNVMEEGKDFQPSRSTAQQELDGKPASPTPVIVASHTANKEEKSLLELEVDLDNLELEDIDTTDINLDEDILDD

SEQ ID NO: 38 - protein, 461 aa, Homo sapiens [sequence omitted]

SEQ ID NO: 39 - protein, 596 aa, Homo sapiens
MSNYSVSLVGPAPWGFRLQGGKDFNMPLTISSLKDGGKAAQANVRIGDVVLSIDGINAQGMTHLEAQNKIKGCTGSLNMTLQRASAAPKPEPVPVQKGEPKEVVKPVPITSPAVSKVTSTNNMAYNKAPRPFGSVSSPKVTSIPSPSSAFTPAHATTSSHASPSPVAAVTPPLFAASGLHANANLSADQSPSALSAGKTAVNVPRQPTVTSVCSETSQELAEGQRRGSQGDSKQQNGPPRKHIVERYTEFYHVPTHSDASKKRLIEDTEDWRPRTGTTQSRSFRILAQITGTEHLKESEADNTKKANNSQEPSPQLASSVASTRSMPESLDSPTSGRPGVTSLTAAAAFKPVGSTGVIKSPSWQRPNQGVPSTGRISNSATYSGSVAPANSALGQTQPSDQDTLVQRAEHIPAGKRTPMCAHCNQVIRGPFLVALGKSWHPEEFNCAHCKNTMAYIGFVEEKGALYCELCYEKFFAPECGRCQRKILGEVISALKQTWHVSCFVCVACGKPIRNNVFHLEDGEPYCETDYYALFGTICHGCEFPIEAGDMFLEALGYTWHDTCFVCSVCCESLEGQTFFSKKDKPLCKKHAHSVNF

SEQ ID NO: 40 - protein, 331 aa, Homo sapiens
MARGGRGRRLGLALGLLLALVLAPRVLRAKPTVRKERVVRPDSELGERPPEDNQSFQYDHEAFLGKEDSKTFDQLTPDESKERLGKIVDRIDNDGDGFVTTEELKTWIKRVQKRYIFDNVAKVWKDYDRDKDDKISWEEYKQATYGYYLGNPAEFHDSSDHHTFKKMLPRDERRFKAADLNGDLTATREEFTAFLHPEEFEHMKEIVVLETLEDIDKNGDGFVDQDEYIADMFSHEENGPEPDWVLSEREQFNEFRDLNKDGKLDKDEIRHWILPQDYDHAQAEARHLVYESDKNKDEKLTKEEILENWNMFVGSQATNYGEDLTKNHDEL

SEQ ID NO: 41 - protein, 1411 aa, Homo sapiens
MLRRILQRTPGRVGSQGSDLDSSATPINTVDVNNESSSEGFICPQCMKSLGSADELFKHYEAVHDAGNDSGHGGESNLALKRDDVTLLRQEVQDLQASLKEEKWYSEELKKELEKYQGLQQQEAKPDGLVTDSSAELQSLEQQLEEAQTENFNIKQMKDLFEQKAAQLATEIADIKSKYDEERSLREAAEQKVTRLTEELNKEATVIQDLKTELLQRPGIEDVAVLKKELVQVQTLMDNMTLERERESEKLKDECKKLQSQYASSEATISQLRSELAKGPQEVAVYVQELQKLKSSVNELTQKNQTLTENLLKKEQDYTKLEEKHNEESVSKKNIQATLHQKDLDCQQLQSRLSASETSLHRIHVELSEKGEATQKLKEELSEVETKYQHLKAEFKQLQQQREEKEQHGLQLQSEINQLHSKLLETERQLGEAHGRLKEQRQLSSEKLMDKEQQVADLQLKLSRLEEQLKEKVTNSTELQHQLDKTKQQHQEQQALQQSTTAKLREAQNDLEQVLRQIGDKDQKIQNLEALLQKSKENISLLEKEREDLYAKIQAGEGETAVLNQLQEKNHTLQEQVTQLTEKLKNQSESHKQAQENLHDQVQEQKAHLRAAQDRVLSLETSVNELNSQLNESKEKVSQLDIQIKAKTELLLSAEAAKTAQRADLQNHLDTAQNALQDKQQELNKITTQLDQVTAKLQDKQEHCSQLESHLKEYKEKYLSLEQKTEELEGQIKKLEADSLEVKASKEQALQDLQQQRQLNTDLELRATELSKQLEMEKEIVSSTRLDLQKKSEALESIKQKLTKQEEEKKILKQDFETLSQETKIQHEELNNRIQTTVTELQKVKMEKEALMTELSTVKDKLSKVSDSLKNSKSEFEKENQKGKAAILDLEKTCKELKHQLQVQMENTLKEQKELKKSLEKEKEASHQLKLELNSMQEQLIQAQNTLKQNEKEEQQLQGNINELKQSSEQKKKQIEALQGELKIAVLQKTELENKLQQQLTQAAQELAAEKEKISVLQNNYEKSQETFKQLQSDFYGRESELLATRQDLKSVEEKLSLAQEDLISNRNQIGNQNKLIQELKTAKATLEQDSAKKEQQLQERCKALQDIQKEKSLKEKELVNEKSKLAEIEEIKCRQEKEITKLNEELKSHKLESIKEITNLKDAKQLLIQQKLELQGKADSLKAAVEQEKRNQQILKDQVKKEEEELKKEFIEKEAKLHSEIKEKEVGMKKHEENEAKLTMQITALNENLGTVKKEWQSSQRRVSELEKQTDDLRGEIAVLEATVQNNQDERRALLERCLKGEGEIEKLQTKVLELQRKLDNTTAAVQELGRENQSLQIKHTQALNRKWAEDNEVQNCMACGKGFSVTVRRHHCRQCGNIFCAECSAKNALTPSSKKPVRVCDACFNDLQG

SEQ ID NO: 42 - protein, 360 aa, Homo sapiens
MSKQQPTQFINPETPGYVGFANLPNQVHRKSVKKGFEFTLMVVGESGLGKSTLINSLFLTDLYPERVIPGAAEKIERTVQIEASTVEIEERGVKLRLTVVDTPGYGDAINCRDCFKTIISYIDEQFERYLHDESGLNRRHIIDNRVHCCFYFISPFGHGLKPLDVAFMKAIHNKVNIVPVIAKADTLTLKERERLKKRILDEIEEHNIKIYHLPDAESDEDEDFKEQTRLLKASIPFSVVGSNQLIEAKGKKVRGRLYPWGVVEVENPEHNDFLKLRTMLITHMQDLQEVTQDLHYENFRSERLKRGGRKVENEDMNKDQILLEKEAELRRMQEMIARMQAQMQMQMQGGDGDGGALGHH

SEQ ID NO: 43 - protein, 300 aa, Homo sapiens
MILLEVNNRIIEETLALKFENAAAGNKPEAVEVTFADFDGVLYHISNPNGDKTKVMVSISLKFYKELQAHGADELLKRVYGSFLVNPESGYNVSLLYDLENLPASKDSIVHQAGMLKRNCFASVFEKYFQFQEEGKEGENRAVIHYRDDETMYVESKKDRVTVVFSTVFKDDDDVVIGKVFMQEFKEGRRASHTAPQVLFSHREPPLELKDTDAAVGDNIGYITFVLFPRHTNASARDNTINLIHTFRDYLHYHIKCSKAYIHTRMRAKTSDFLKVLNRARPDAEKKEMKTITGKTFSSR

SEQ ID NO: 44 - protein, 425 aa, Homo sapiens
MAVAVGRPSNEELRNLSLSGHVGFDSLPDQLVNKSTSQGFCFNILCVGETGIGKSTLMDTLFNTKFESDPATHNEPGVRLKARSYELQESNVRLKLTIVDTVGFGDQINKDDSYKPIVEYIDAQFEAYLQEELKIKRSLFNYHDTRIHACLYFIAPTGHSLKSLDLVTMKKLDSKVNIIPIIAKADTIAKNELHKFKSKIMSELVSNGVQIYQFPTDEETVAEINATMSVHLPFAVVGSTEEVKIGNKMAKARQYPWGVVQVENENHCDFVKLREMLIRVNMEDLREQTHTRHYELYRRCKLEEMGFKDTDPDSKPFSLQETYEAKRNEFLGELQKKEEEMRQMFVMRVKEKEAELKEAEKELHEKFDLLKRTHQEEKKKVEDKKKELEEEVNNFQKKKAAAQLLQSQAQQSGAQQTKKDKDKKK

(19) United States
(12) Patent Application Publication
(10) Pub. No.: US 2016/0199413 A1
(43) Pub. Date: Jul. 14, 2016

(54) MSCS IN THE TREATMENT OF INFLAMMATORY PULMONARY DISEASES

(71) Applicant: ISLETONE AB, Hägersten (SE)

(72) Inventors: Oscar SIMONSON, Stockholm (SE); Katarina LE BLANC, Sköndal (SE); Henrik JOHANSSON, Solna (SE); Karl-Henrik GRINNEMO, Hägersten (SE)

(73) Assignee: ISLETONE AB, Hägersten (SE)

(21) Appl. No.: 14/909,027

(22) PCT Filed: Jul. 31, 2014
(86) PCT No.: PCT/SE2014/000103

§ 371 Date: Jan. 29, 2016

(30) Foreign Application Priority Data
Aug. 1, 2013 (SE) 1300521-0

(51) Int. Cl. A61K 35/28; C12N 5/0775; A61K 35/16

(52) U.S. Cl. CPC A61K 35/28; A61K 35/16; C12N 5/0663

(57) ABSTRACT

The present invention relates to, inter alia, methods for culturing mesenchymal stem cells (MSCs) (without the use of non-human serum components), MSCs and extracellular vesicles obtained from such culturing methods, having advantageous polypeptide profiles which are key for immuno-modulatory efficacy. Further, the present invention pertains to pharmaceutical compositions comprising such MSCs and/or extracellular vesicles, and medical treatment and prophylactic methods and medical uses of MSCs and/or extracellular vesicles in a variety of diseases, notably acute respiratory distress syndrome (ARDS), infant respiratory distress syndrome (IRDS), pulmonary hypertension (PH), and related diseases and ailments, such as organ failure.

### TECHNICAL FIELD

The present invention relates to, inter alia, methods for culturing mesenchymal stem cells (MSCs) (without the use of non-human serum components), MSCs obtained from such culturing methods, MSCs and/or extracellular vesicles derived from said MSCs having proteomic profiles which ensure immuno-modulatory capacity, pharmaceutical compositions comprising such MSCs, and medical treatment and prophylactic methods and medical uses of MSCs and/or extracellular vesicles in a variety of diseases.

### BACKGROUND ART

Mesenchymal stem cells (MSCs) can be found in inter alia the bone marrow, blood, dermis, periosteum and various other tissues and MSCs can differentiate into a variety of cell types, including adipose, areolar, osseous, cartilaginous, elastic, marrow stroma, muscle, fibrous connective tissue, and cardiac tissue, depending upon external milieu and stimulants. Due to their cellular origin and their phenotype, MSC do normally not stimulate adverse immune responses, which enables using unrelated human donors in clinical settings.

MSCs are normally isolated from the tissue, purified, and then expanded in an appropriate culture medium. Suitable culture medium for MSCs may comprise various components to promote MSC expansion, for instance growth factors, cytokines, and serum. After the isolation, purification, and culture expansion steps, the MSCs are washed and centrifuged, followed by freezing in a suitable cryopreservation medium. At the time of treatment, the MSCs are thawed just prior to administration to a patient. The prior art normally discloses expansion processes involving cell culturing in the presence of non-human components (commonly fetal bovine serum (FBS)).

Xenogeneic proteins deriving from the non-human serum may elicit cell-mediated or humoral immune responses (e.g., the generation of anti-bovine serum protein antibodies), which may result in less efficient engraftment of the MSCs, particularly if such xenogeneic proteins become associated with MSC cell-surface membranes. Media comprising autologous human serum have been suggested but this approach has been considered unfeasible when the quantities of cells required in the ultimate MSC product exceed that which can be grown in a fixed amount of autologous serum. Additionally, the use of autologous human serum presupposes that the patient will have sufficient time and be in sufficient health to donate serum in advance of the initiation of MSC therapy. The current conventional MSC culturing process typically requires 2 to 10 weeks to isolate, expand, harvest and purify a suitable number of cells to constitute a pharmaceutical treatment. In some cases a pharmaceutical treatment consists of 1 dose. In other cases a pharmaceutical treatment consists of 2 or more doses. Unfortunately, in some cases, MSC therapy is needed less than about 2 weeks from diagnosis or presentation of clinical symptoms, or in less than about 1 week from diagnosis or presentation of clinical symptoms, or in less than about 48 hours of diagnosis or presentation of clinical symptoms. When MSC therapy is needed within a short time period from diagnosis or presentation of clinical symptoms, MSCs that have already been manufactured, purified and cryopreserved exhibit the significant benefit of being available upon diagnosis or presentation of an acute illness. However, determining clinical therapeutic potency of MSCs remains a challenge.

In vitro findings indicate that MSC are immunosuppressive, and rodent, baboon or human MSCs suppress lymphocyte proliferation in mixed lymphocyte cultures, as well as inhibiting the formation of cytotoxic T-cells and NK-cells. The unique immunoregulatory and regenerative properties of MSCs make them an attractive tool for cellular treatment of autoimmunity and inflammation and MSCs have been applied in a variety of clinical contexts, for instance in the treatment of severe graft-versus-host disease (Le Blanc, 2004, Lancet), but also for treating multiple sclerosis and Crohn's disease. However, there are numerous other severe indications for which there is currently a lack of efficient medical treatments, and the present invention thus aims to expand the usage of MSCs, obtained from cell cultures devoid of non-human animal products, to additional indications where there is an unmet medical need. Furthermore, although MSCs are being investigated in several ongoing clinical trials very little is known about what makes MSCs clinically effective, i.e. which characteristics that are necessary for MSCs to be capable of exerting the desired therapeutic effects in a given disease.

### SUMMARY OF THE INVENTION

The present invention relates to, inter alia, methods for culturing mesenchymal stem cells (MSCs) (without the use of non-human serum components), methods for selecting clinically potent MSCs and/or extracellular components (primarily extracellular vesicles such as exosomes) for the treatment of respiratory inflammation and associated conditions, MSCs and/or extracellular vesicles having clinically efficacious proteomics profiles and immuno-modulatory capacity obtained from such culturing and/or selection methods, pharmaceutical compositions comprising such MSCs and/or vesicles, and medical treatment and prophylactic methods and medical uses of MSCs in a variety of diseases, notably acute respiratory distress syndrome (ARDS) and associated conditions.

In one aspect, the present invention relates to a method for culturing mesenchymal stem cells (MSCs), comprising culturing MSCs (of a suitable origin) in a culture medium comprising lyzed human trombocytes, as well as, in additional aspects, selection methods and immuno-modulation criteria, and cell culture compositions for culturing MSCs and MSCs obtainable by the methods of the present invention. Further, in yet other aspects, the instant invention pertains to MSCs and extracellular vesicles thereof as such, pharmaceutical compositions, methods for preparing said pharmaceutical compositions, and medical uses involving MSCs and extracellular vesicles, as well as treatment methods involving MSCs.

In one aspect, the present invention relates to methods for selecting clinically effective immuno-modulatory MSCs and related paracrine factors (e.g. extracellular vesicles such as exosomes), by utilizing proteomics profiling and by assessing the functional characteristics of the MSCs. More specifically, the MSCs of the present invention display certain characteristics in terms of polypeptide expression as well as in terms of polypeptide expression in extracellular fractions (which may comprise extracellular vesicles such as exosomes and, additionally, extracellular polypeptides).

In a further aspect, the instant invention pertains to MSCs and/or extracellular vesicles obtainable by the methods according to the present invention.

In another aspect, the present invention relates to a cell culture composition for culturing MSCs, wherein the composition comprises a cell culture medium (for instance Dulbecco's Modified Eagles Medium (DMEM)) and lyzed human trombocytes.

In yet another aspect, the present invention pertains to a method of preparing a pharmaceutical composition comprising MSCs, comprising the steps of culturing MSCs (preferably of crista iliaca origin and/or sternum origin) in a cell culture composition comprising lyzed human trombocytes, passaging the MSCs in the culture not more than 5 times, and, re-suspending the MSCs in saline solution to a final concentration of between approximately 5×10⁵ and 4×10⁶ MSCs per ml.

In a another aspect, the instant invention pertains to pharmaceutical compositions in accordance with the present invention for use in medicine, specifically, in a seventh aspect, for use in the treatment and/or prophylaxis of acute respiratory distress syndrome (ARDS), infant respiratory distress syndrome (IRDS), pulmonary hypertension (PH), and/or acute organ failure (for instance kidney, liver, and/or heart failure) in connection with ARDS or IRDS.

In yet another aspect, the present invention relates to a mesenchymal stem cell (MSC), wherein the MSCs display a spindle-shape morphology and expressing CD73, CD90, and CD105, and wherein the MSCs are devoid of CD34, CD45, CD14, and CD3. The MSCs may be either positive or negative for HLA-ABC (MHC class I) and/or HLA-DR (MHC class II), for instance depending on the degree of priming/activation of the MSCs. Further, the MSCs may be negative for CD11b, CD19, and/or CD31.

In a further aspect, the instant invention pertains to a method of treating and/or preventing diseases and/or disorders selected from the group comprising acute respiratory distress syndrome (ARDS), infant respiratory distress syndrome (IRDS), pulmonary hypertension (PH), and acute organ (for instance kidney, liver, and/or heart) failure (optionally in connection with ARDS or IRDS), comprising the steps of providing a therapeutic dose of MSCs, and administering the therapeutic dose to a patient suffering from any one of the abovementioned diseases.

Thus the present invention provides a novel therapeutic modality, based on MSCs displaying a specific proteomics profile, potency in in vitro immuno-modulatory assays, cultured via surprisingly advantageous methods, for the treatment of various illnesses where there is currently a significant unmet medical need, notably ARDS, IRDS, PH, and related disorders and illnesses. The polypeptide profiling and the proven immuno-modulatory activity result in MSCs that are clinically effective, in stark contrast to many MSC preparations described in the art. Furthermore, the absence of non-human components during the culturing processes and the advantageous components comprised in the cell culture compositions and in the pharmaceutical compositions enable highly efficacious clinical treatment of these severe illnesses.

### BRIEF DESCRIPTION OF THE DRAWINGS

FIG. 1. CRP decreased strongly after administration of the MSCs.

FIG. 2. Bronchoalveolar lavage (BAL) interleukin-6 (IL-6) decreased post-treatment, evidencing decreased pulmonary inflammation.

FIG. 3. Partial oxygen pressure (pO₂) increased significantly from pre-injection to day 3 post-injection and partial carbon dioxide pressure (pCO₂) displayed a concomitant decrease.

FIG. 4. Expiratory tidal volume increased in response to MSC administration.

FIG. 5. Pulmonary compliance increased significantly post MSC treatment.

FIG. 6. Serum levels of RANTES were clearly elevated approximately four weeks after treatment, indicating stem cell mobilization in response to the MSC injection.

FIG. 7. Serum creatinine levels decreased considerably upon MSC injection in a patient with acute kidney failure induced by ARDS

FIGS. 8 and 9. Lung X-rays evidencing significant pulmonary recovery.

FIG. 10. Plasma surfactant B levels increased substantially post-treatment, showing recovery of pneumocytes type 2.

FIG. 11. (A) In line with the consensus statement of the International Society for Cellular Therapy (ISCT) infused bone marrow-derived MSCs expressed CD73, CD90, and CD105 and lacked expression of CD14, CD34, CD45, and HLA-DR as assessed by flow cytometry (FACS) and shown as histograms. Furthermore, MSCs were negative for the endothelial marker CD31.

FIG. 12. Inflammatory cytokines can activate (“prime”) MSCs, which is considered a key step (also known as “licensing”) for MSCs to efficiently control inflammation. The characteristic up-regulation of ICAM-1 (CD54), VCAM-1 (CD106), HLA-ABC, and HLA-DR upon 48 hours of cell exposure towards the pro-inflammatory IFN-γ and TNF-α was confirmed by FACS on the MSCs.

FIG. 13. MSCs promote in vitro generation of regulatory T-cells. MSCs are capable of promoting the induction and expansion of immune suppressive regulatory T-cells (T_{Regs}). Co-culturing MSCs in different ratios with purified healthy control T-cells led to an increase of the CD25⁺CD127^{dim} T_{Reg}-fraction among the CD4⁺ T-cells. Control T-cells were stimulated using activating anti-CD3 and anti-CD28 microbeads. The left panel shows a representative flow cytometry (FACS) dot plot analysis and the right panel the mean values (columns) of two independent experiments performed in duplicates.

FIG. 14. In selected experiments MSCs were exposed to IFN-γ and TNF-α for 48 hours (primed MSCs, pMSCs). After priming, IDO expression was substantially up-regulated as assessed by quantitative PCR (A). Unstimulated MSC and pMSC affected viability of control or LPS-stimulated PMN as demonstrated in panel B. (C) CD16 (FcγR-III) expression was demonstrated to be used as a marker of PMN viability and matched with the percentage of viable PMNs in all culture conditions. Similarly, CD11b and CD54 expressions were associated with PMN activation status. The percentage of CD11b-positive PMNs was higher in the presence of MSCs and further enhanced by LPS treatment (D), while CD54 relative mean fluorescence intensity (rMFI) was up-regulated by LPS treatment and this effect was enhanced by co-culture with MSCs (E). Next, the capacity of MSC to promote the generation of myeloid-derived suppressor cells (MDSC) was tested within our co-culture model with PMNs. Co-culture with MSCs led to a marked increase of mature CD11^{bright}/CD16^{bright}/CD62L^{dim} (N2-type) PMNs resembling granulocytic MDSCs (F). (G) MSC and PMN co-cultures were stained with May Grunwald-Giemsa dye to observe cell morphology following reciprocal interaction for 24 hours. According to the marked increase of mature CD11^{bright}/CD16^{bright}/CD62L^{dim} (N2-type) PMNs shown at this time by flow-cytometry, PMNs displayed a hypersegmented nuclear morphology after co-culture with both resting (PMN MSC 24 h) and primed MSCs (PMN pMSC 24 h). Similarly, co-culturing MSCs with healthy control peripheral blood mononuclear cells (PBMCs) at different ratios promoted CD14⁺HLA-DR^{low} monocytes resembling monocytic MDSCs (H). MSCs were also capable of promoting the induction and expansion of immune suppressive CD4⁺CD25^{high}CD127^{low} regulatory T-cells (T_{Regs}), when co-cultured in different ratios with purified healthy control T-cells (I). (J) Increased levels of circulating CD4⁺CD25^{high}CD127^{low} T_{Regs}, were also observed up to 20 days following MSC administration. (K) This could in part result from increased thymic output as indicated by increased proportion of CD31⁺ recent thymic emigrants (RTE) among CD45RA⁺ naïve T_{regs} in patients. Bars indicate the standard error mean. Abbreviations: *p-value≦0.05, **p-value≦0.01, ***p-value≦0.01.

FIG. 15. Panel A demonstrates the disease progression and concomitant chest-X-rays (CXRs) from the onset of influenza A H1N1, until hospital discharge. As demonstrated, there was a progression of the bilateral opacifications of the lungs after the onset of the influenza infection, and the patient required both mechanical ventilation and ECMO support. By 24 hours after MSC infusion, there was a decrease in pulmonary infiltrates on CXR, followed by progressive improvement until the time of weaning of the ECMO and extubation 4 weeks after MSC administration. In parallel with the improvement in CXRs, both the pulmonary compliance (B) and tidal volumes (C) improved back to normal levels within the first 2 days. However, 5 days after MSC administration, the patient developed a nosocomial pneumonia with increased white blood cell (WBC) count (D), and new infiltrates on CXR (A), which resolved within 3 days. Liver function also improved during the first week with normalization of serum ALT, AST and bilirubin levels (E).

FIG. 16. Panel A demonstrates the progression of ARDS both on CXRs and serial computed tomographic scanning. As demonstrated, there was progressive decrease in pulmonary infiltrates starting the first day after MSC infusion. By day 7, the CXR improved back to normal and the patient was weaned off ECMO the following day and extubation at day 12. (B) Pulmonary compliance and (C) tidal volumes improved back to normal levels during the first week. After MSC infusion, the bone-marrow function continued to be reconstituted with resolution of (D) leukopenia and (E) thrombocytopenia.

FIG. 17. The cytokine response in BAL (A) Levels of caspase-cleaved cytokeratin (ccK)-18, indicative of epithelial apoptosis, and of uncleaved cytokeratin (K)-18, indicative of total epithelial death in BAL fluid, decreased in both patients within a few hours after MSC administration. (B) In parallel, surfactant protein B (SP-B) increased during the first 3 days after MSC infusion, suggesting recovery of the alveolar epithelial function (C). Several miRNAs related to inflammation demonstrated a significant decline within the first 24 hours after MSC administration. A transient increase in these miRNA levels was observed by day 7 in patient one due to pneumonia and returned to normal by day 28. As compared to healthy donors.

### DETAILED DESCRIPTION OF THE INVENTION

The present invention relates to, inter alia, methods for culturing mesenchymal stem cells (MSCs) (without the use of non-human serum components), methods for selecting clinically potent immuno-modulatory MSCs and/or extracellular components (primarily extracellular vesicles such as exosomes) for the treatment of respiratory inflammation and associated conditions, MSCs and/or extracellular vesicles having clinically efficacious proteomics profiles obtained from such culturing and/or selection methods, pharmaceutical compositions comprising such MSCs and/or vesicles, and medical treatment and prophylactic methods and medical uses of MSCs in a variety of diseases, notably acute respiratory distress syndrome (ARDS) and associated conditions.

Where features, embodiments, or aspects of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects, alternatives, and/or embodiments of the invention. For instance, aspects, alternatives, and/or embodiments described in connection with the methods for obtaining clinically effective immuno-modulatory MSCs (e.g. the immuno-modulation critera) may also be applicable, relevant, and/or combined with teachings pertaining to, for instance, embodiments relating to the MSCs and/or extracellular vesicles obtainable by said methods. By way of another non-limiting example, aspects, alternatives, and/or embodiments described in connection with the method for culturing MSCs in the absence of non-human serum components may also be applicable to other aspects and/or embodiments as per the present invention, for instance the immuno-modulation selection criteria, meaning that the present invention although not explicitly mentioned herein also encompasses combing the teachings relating to, for instance, the culturing methods with the teachings of the immuno-modulation selection methods. Similarly, aspects, alternatives, and/or embodiments described in connection with the MSCs and/or the extracellular vesicles may naturally also be applicable, relevant, and/or combined with teachings pertaining to the pharmaceutical compositions per se, which comprises said MSCs and/or extracellular vesicles.

Generally, all polypeptides and/or nucleotides disclosed in the present application naturally encompass polypeptide and/or nucleotide sequences that have at least 50% sequence identity to the polypeptide and/or nucleotide in question, preferably 70% sequence identity to the polypeptide and/or nucleotide in question, more preferably a sequence identity of at least 80%, and even more preferably a sequence identify of at least 90% to the polypeptide and/or nucleotide in question.

For convenience and clarity, certain terms employed herein are collected below. Unless otherwise defined, all technical and scientific terms used herein have the same meaning as commonly understood by one of ordinary skill in the art to which this invention belongs.

The terms “positive for” and “negative for” in the context of the present invention (e.g. an MSC which is “positive for” a polypeptide and/or polynucleotide in question) shall be understood in accordance with the meaning normally given to the term within the biological and medical sciences, in essence a cell that is positive for a certain polypeptide and/or polynucleotide expresses said polypeptide and/or polynucleotide. The polypeptide and/or polynucleotide in question may be identified via various means, for instance using fluorescence-activated cell sorting (FACS) and/or immunohistochemical techniques and/or proteomics techniques such as LC-MS and/or 2D-PAGE. The term “positive for” may in certain instances be understood to comprise cell populations where at least 50% of the cells express the polypeptide (or polynucleotide or any other marker) in question, but preferably at least 70% or even more preferably at least 90% of the population expresses the polypeptide in question. The term “negative for” may, in the same vein, naturally be understood to be the opposite of the term “positive for”, i.e. at least 50% —but preferably at least 70% or even more preferably at least 90% —of the cells of the population shall not express the polypeptide (or other suitable marker) in question.

The term “population”, which may relate to MSCs or to extracellular vesicles such as exosomes, shall be understood to encompass a plurality of entities constituting a given population, for instance the individual MSCs which when present in a plurality constitute an MSC population. Thus, naturally, the present invention pertains also to the individual cells and vesicles of e.g. an MSC population or a population of extracellular vesicles, respectively.

The terms “subject” and/or “individual” and/or “patient” may be used interchangeably herein and are to be understood to refer broadly to an animal, for instance a human being, from whom cells can be obtained and/or to whom treatment, including prophylaxis or preventative treatment (for instance using the cells as per the present invention) is provided. Advantageously, the subject of the treatments as described in the context of the present invention is a mammal, preferably a human, or other mammals, preferably domesticated or production mammals.

The term “therapeutically effective amount” is to be understood to refer to an amount which results in an improvement, allevation, or remediation of the disease, disorder, or symptoms of the disease or condition.

The terms “administering,” “introducing” and “transplanting” are used interchangeably for the purposes of the present invention, for instance in the context of the administration of the MSCs to a patient suffering from any of the diseases and/or disorders mentioned in the context of the present invention. A suitable method or route is one which leads to at least partial localization of the MSCs at a desired site. The cells may be administered (delivered) by any appropriate route which results in delivery of the cells and/or paracrine factors excreted from the cells to a desired location/tissue/site in the subject. The modes of administration suitable for the purposes of the present invention comprise for instance (without limitation) intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrase “pharmaceutically acceptable excipient” as used herein is to be understood to relate to a pharmaceutically acceptable material, composition or vehicle, for instance a solid or liquid filler, a diluent, an excipient, a carrier, a solvent or an encapsulating material, involved in suspending, maintaining the activity of or carrying or transporting the subject agents from one organ, or portion of the body, to another organ, or portion of the body.

In one aspect, the present invention pertains to methods for obtaining immuno-modulatory MSCs. Ensuring that MSCs have clinically effective immuno-modulatory capacity is a crucial but in the prior art frequently overlooked aspect. The present invention relates to methods for validating the immuno-modulatory properties of MSCs by methods comprising the steps of (i) culturing MSCs (ii) establishing that the MSC population fulfils at least one of the following immuno-modulation criteria:
(a) the MSC population is positive for at least one of the following polypeptides: vimentin (SEQ ID No 1), caldesmon (SEQ ID No 2), annexin A1 (SEQ ID No 3), 14-3-3 protein epsilon (SEQ ID No 4), ADP ribosylation factor 1 (SEQ ID No 5), calnexin (SEQ ID No 6), ADP ribosylation factor 5 (SEQ ID No 7), transforming protein RhoA (SEQ ID No 8), CD44 (SEQ ID No 9), coactosin-like protein (SEQ ID No 10), mitogen-activated protein kinase 3 (SEQ ID No 11), insulin-like growth factor-binding protein 7 (SEQ ID No 12), N-acetyl-glucosamine-6-sulfatase (SEQ ID No 13), cellular retinoic acid-binding protein 2 (SEQ ID No 14), transcription elongation factor B polypeptide 1 (SEQ ID No 15), NEDD8 (SEQ ID No 16), fatty acid-binding protein, heart (SEQ ID No 17);
(b) a population of extracellular vesicles derived from the MSC population is positive for at least one of the following polypeptides: serotransferrin (SEQ ID No 18), versican core protein (SEQ ID No 19), annexin A2 (SEQ ID No 20), serine protease HTRA1 (SEQ ID No 21), insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerase (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34);
(c) the MSC population in (a) displays the following order of polypeptide abundance: vimentin>Annexin A1. Preferably, the MSC population exhibits the following order of polypeptide abundance: vimentin>Annexin A1>CD44>insulin-like growth factor binding protein 7>fatty-acid binding protein 3;
(d) the extracellular vesicle population in (b) displays the following order of polypeptide abundance: serotransferrin>annexin A2. Preferably, the extracellular vesicle population displays the following polypeptide abundance: serotransferrin>annexin A2>connective tissue growth factor
(e) the fold-increase expression of indoleamine 2,3-dioxygenase (IDO) in the MSC population is <10 when the MSCs are primed with 15 ng/mL TNF-alpha;
(f) the fold-increase expression of indoleamine 2,3-dioxygenase (IDO) in the MSC population is >100 when the MSCs are primed with 10 ng/mL IFN-gamma;
(g) the viability of polymorphonuclear neutrophils (PMNs) is increased by at least 20% (preferably at least 30% or even more preferably at least 50%) when co-cultured with MSCs from the MSC population primed with IFN-gamma or TNF-alpha in accordance with (e) or (f);
(h) the number of CD14⁺HLA-DR^{low} monocytes is increased at least 1.5-fold (more preferably at least 2-fold, or even more preferably at least 3-fold) when healthy control human peripheral blood mononuclear cells (PBMCs) are co-cultured with the MSC population primed with IFN-gamma or TNF-alpha in accordance with (e) or (f);
(i) the number of CD4⁺CD25^{high}CD127^{low} regulatory T-cells (T_{Regs}) is increased at least 1.5-fold (more preferably at least 2-fold, or even more preferably at least 3-fold) when healthy control human peripheral blood mononuclear cells (PBMCs) are co-cultured with the MSC population primed with IFN-gamma or TNF-alpha in accordance with (e) or (f).

Furthermore, the extracellular vesicle population obtainable from the MSC population may preferably be negative for (i.e. devoid of) at least one of the following polypeptides: LIM domain only protein7 (SEQ ID No 35), LIM domain and actin-binding protein 1 (SEQ ID No 36), coatomer protein complex, subunit beta 2 (Beta prime), isoform CRA_b (SEQ ID No 37), ribonuclease inhibitor (SEQ ID No 38), PDZ and LIM domain protein 5 (SEQ ID No 39), reticulocalbin-1 (SEQ ID No 40), early endosome antigen 1 (SEQ ID No 41), septin-2 (SEQ ID No 42), actin-related protein 2/3 complex subunit 2 (SEQ ID No 43), septin 11 (SEQ ID No 44).

Naturally, the MSC culture (i.e. the MSC population, the population of extracellular vesicles derived from the MSCs, and both the MSC population and the population of extracellular vesicles derived from the MSCs) may fulfil only one (1) of the above criteria, but preferably several criteria are met. For instance, the culture may meet criteria (a) and (b), (a) and (c), (b) and (c), (a) and (d), (a) and (e), (b) and (d), (a) and (e), (a), (b), and (c), (a), (b), and (d), (b), (c), and (d), (c), (d), and (e), (d) and (e), (a), (f), and (g), (e), (f), and (g), (a), (b), and (g), (a), (c), and (f), etc., etc. Thus, the MSC culture may meet all possible combinations and permutations of the above criteria (a)-(i) without departing from the scope of the present invention.

In essence, based on screening of a large number of MSC cultures the inventors have realized that certain polypeptide profiles of both cells and extracellular components contribute strongly to the therapeutic immuno-modulatory efficacy. Some of the key polypeptide expression features are summarized in the tables below but other polypeptide expression patterns in addition to the ones explicitly mentioned have also been linked to immuno-modulatory properties.

Polypeptide Expression-Active MSC-derived Extracellular Vesicles

| Preferably positive | SEQ | Preferably negative for | SEQ |
|---|---|---|---|
| for at least one of: | ID No | at least one of: | ID No |
| Serotransferrin | 18 | LIM domain only protein 7 | 35 |
| Versican core protein | 19 | LIM domain and actin-binding | 36 |
| | | protein 1 | |
| Annexin A2 | 20 | Coatomer protein complex, | 37 |
| | | subunit beta 2 (Beta prime), | |
| | | isoform CRA_b | |
| Serine protease HTRA1 | 21 | Ribonuclease inhibitor | 38 |
| IGFBP3 | 22 | PDZ and LIM domain | 39 |
| | | protein 5 | |
| Connective tissue GF | 23 | Reticulocalbin-1 | 40 |
| Vinculin | 24 | Early endosome antigen 1 | 41 |
| Neuroblast differentiation- | 25 | Septin-2 | 42 |
| associated protein AHNAK | | | |
| Microtubule-associated | 26 | Actin-related protein 2/3 | 43 |
| protein | | complex subunit 2 | |
| Fatty acid-synthase | 27 | Septin 11 | 44 |
| Triosephosphate isomerise | 28 | | |
| ATP-citrate synthase | 29 | | |
| Calreticulin | 30 | | |
| Vigilin | 31 | | |
| DNA-dependent protein | 32 | | |
| kinase catalytic subunit | | | |
| Rab GDP dissociation | 33 | | |
| inhibitor beta | | | |
| ATP synthase subunit | 34 | | |
| beta, mitochondrial | | | |

Polypeptide Abundance Patterns (Higher-to-Lower) of
Active MSCs & Extracellular Vesicles

| MSCs | Extracellular Vesicles |
|---|---|
| Vimentin < Annexin A1 | Serotransferrin < Annexin A2 |
| Vimentin < Caldesmon | Serotransferrin < Versican core protein |
| Vimentin < Transforming | Serotransferrin < Annexin A2 < |
| protein RhoA | Connective tissue growth factor |
| Vimentin < CD44 | Serotransferrin < Vinculin |
| Vimentin < Annexin A1 < CD44 | Serotransferrin < Annexin A2 < |
| | Vinculin |

The sources of the MSCs as per the present invention may be selected from the group comprising bone marrow, cord blood, amniotic tissue, Wharton's jelly, tooth bud, adipose tissue, embryonic or fetal material, but various other sources of MSCs could also be applicable. MSCs of bone marrow origin are normally of crista iliaca origin and/or of sternum origin.

Importantly, in order to retain the immuno-modulatory potential of the MSCs (for instance the order of abundance between vimentin and annexin A1, or any of the other abundance patterns of the MSCs and/or the extracellular vesicles referred to above), the MSCs shall preferably not be passaged more than 5 times before clinical use.

In yet another aspect, the present invention pertains to a population of immuno-modulatory MSCs obtainable by the methods of the present invention.

In a further aspect, the present invention relates to a population of immuno-modulatory MSCs having the following antigen profile: CD73+, CD90+, CD105+, CD34−, CD45−, CD14−, and CD3−. In a further embodiment, the MSC population may be positive for vimentin and/or Annexin A1, and further positive for insulin-like growth factor binding protein 7 and/or fatty-acid binding protein 3 and/or Annexin A1.

MSCs are normally defined as functional and biologically active through a colony unit forming (CFU) test and differentiation into adipocytes (fat cells), osteoblasts (bone cells), and chondrocytes (cartilage cells). Madeira and co-authors (PLOS One, 2012) have compared biologically active and inactive MSCs and found that in inactive cells, expression of annexin A1 is upregulated 1.5 fold. Annexin A1 is a known apoptosis-related protein, which impacts adaptive and innate immunity. In contrast, expression of vimentin, which is a cellular cytoskeleton component, is downregulated 2.5 fold in biologically inactive MSCs in comparison with that in the active ones. This probably reflects downregulation in proliferation capacity of the inactive MCSs. However, what the inventors of the present inventions have unexpectedly found is that it is clearly preferential with a larger abundance of vimentin (and related polypeptides) than of annexin A1 (and related polypeptides). Similarly, other abundance patterns have been shown in accordance with the present invention to result in enhanced immuno-modulatory potency, for instance in the context of the whole cells lysates: Vimentin<Caldesmon, Vimentin<CD44, and Vimentin<Annexin A1<CD44. And, in the context of the lysate of the fraction containing extracellular vesicles: Serotransferrin<Versican core protein, Serotransferrin<Annexin A2<Connective tissue growth factor, and Serotransferrin<Annexin A2<Vinculin. Importantly, the different polypeptide profiles of the MSCs and the extracellular vesicles may co-exist, for instance in that the whole cell lysate (i.e. the MSC polypeptide pattern) may display a greater abundance of vimentin than of Annexin A1, whereas the extracellular vesicle fraction derived from the MSC population exhibits a greater abundance of serotransferrin than of Annexin A2.

Naturally, the abovementioned profiles may be detected/assessed either at the point of obtaining the material from a donor, at various time points during the expansion/culturing of the MSCs prior to clinical application, at various time points after the cells have been cultured in vitro, and/or at the point when it is time to administer the MSCs and/or extracellular vesicles to a patient to be treated.

In a further embodiment, the MSC population may be positive for the CD44 antigen, and CD44 may advantageously be present in a lower abundance than Annexin A1.

As abovementioned, the MSC population in accordance with the present invention meet at least one of the following immuno-modulation critera:
(a) the MSC population is positive for at least one of the following polypeptides: vimentin (SEQ ID No 1), caldesmon (SEQ ID No 2), annexin A1 (SEQ ID No 3), 14-3-3 protein epsilon (SEQ ID No 4), ADP ribosylation factor 1 (SEQ ID No 5), calnexin (SEQ ID No 6), ADP ribosylation factor 5 (SEQ ID No 7), transforming protein RhoA (SEQ ID No 8), CD44 (SEQ ID No 9), coactosin-like protein (SEQ ID No 10), mitogen-activated protein kinase 3 (SEQ ID No 11), insulin-like growth factor-binding protein 7 (SEQ ID No 12), N-acetyl-glucosamine-6-sulfatase (SEQ ID No 13), cellular retinoic acid-binding protein 2 (SEQ ID No 14), transcription elongation factor B polypeptide 1 (SEQ ID No 15), NEDD8 (SEQ ID No 16), fatty acid-binding protein, heart (SEQ ID No 17);
(b) a population of extracellular vesicles derived from the MSC population is positive for at least one of the following polypeptides: serotransferrin (SEQ ID No 18), versican core protein (SEQ ID No 19), annexin A2 (SEQ ID No 20), serine protease HTRA1 (SEQ ID No 21), insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerise (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34);
(c) the MSC population in (a) displays the following order of polypeptide abundance: vimentin>annexin A1;
(d) the extracellular vesicle population in (b) displays the following order of polypeptide abundance: serotransferrin>annexin A2.
(e) the fold-increase expression of indoleamine 2,3-dioxygenase (IDO) in the MSC population is <10 when the MSCs are primed with 15 ng/mL TNF-alpha;
(f) the fold-increase expression of indoleamine 2,3-dioxygenase (IDO) in the MSC population is >100 when the MSCs are primed with 10 ng/mL IFN-gamma;
(g) the viability of polymorphonuclear neutrophils (PMNs) is increased by at least 20% (preferably by at least 30%) when co-cultured with MSCs from the MSC population primed with IFN-gamma or TNF-alpha in accordance with (e) or (f);
(h) the number of CD14⁺HLA-DR^{low} monocytes is increased at least 1.5-fold (preferably 2-fold or 3-fold) when healthy control human peripheral blood mononuclear cells (PBMCs) are co-cultured with the MSC population primed with IFN-gamma or TNF-alpha in accordance with (e) or (f);

(i) the number of CD4⁺CD25^{high}CD127^{low} regulatory T-cells (T_{Regs}) is increased at least 1.5-fold (preferably 2-fold or 3-fold) when healthy control human peripheral blood mononuclear cells (PBMCs) are co-cultured with the MSC population primed with IFN-gamma or TNF-alpha in accordance with (e) or (f).

Similarly, as mentioned in connection with the immuno-modulation criteria above and also as exemplified by e.g. the above tables, the population of the extracellular vesicles obtainable from the MSCs as per the present invention is preferably negative for (i.e. devoid of) the following polypeptides: LIM domain only protein? (SEQ ID No 35), LIM domain and actin-binding protein 1 (SEQ ID No 36), coatomer protein complex, subunit beta 2 (Beta prime), isoform CRA_b (SEQ ID No 37), ribonuclease inhibitor (SEQ ID No 38), PDZ and LIM domain protein 5 (SEQ ID No 39), reticulocalbin-1 (SEQ ID No 40), early endosome antigen 1 (SEQ ID No 41), septin-2 (SEQ ID No 42), actin-related protein 2/3 complex subunit 2 (SEQ ID No 43), septin 11 (SEQ ID No 44).

In yet another aspect, the present invention pertains to a vesicle population of extracellular vesicles derived from the MSC population in accordance with the present invention. The vesicle population preferably comprises extracellular vesicles in the form of exosomes.

As above-mentioned, the MSCs as per the present invention (i.e. the MSC population, the population of extracellular vesicles derived from the MSCs, and both the MSC population and the population of extracellular vesicles derived from the MSCs) may fulfil only one (1) of the above criteria, but preferably several criteria are met. For instance, the culture may meet criteria (a) and (b), (a) and (c), (b) and (c), (a) and (d), (a) and (e), (b) and (d), (a) and (e), (a), (b), and (c), (a), (b), and (d), (b), (c), and (d), (c), (d), and (e), (d) and (e), (a), (f), and (g), (e), (f), and (g), (a), (b), and (g), (a), (c), and (f), etc., etc. Thus, the MSC culture may meet all possible combinations and permutations of the above criteria (a)-(i) without departing from the scope of the present invention.

Naturally, all the expression features and patterns mentioned in connection with the immuno-modulation criteria above also apply to the MSCs and/or the extracellular vesicles as such.

Extracellular vesicles and other paracrine factors are of great importance for the therapeutic efficacy of MSCs and the vesicle population thus meets the above-mentioned immuno-modulation criteria, namely that the vesicle population may be positive for at least one of the following polypeptides: serotransferrin (SEQ ID No 18), versican core protein (SEQ ID No 19), annexin A2 (SEQ ID No 20), serine protease HTRA1 (SEQ ID No 21), insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerise (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34). And, as above-mentioned, the population of extracellular vesicles is preferably negative for (i.e. devoid of) the following polypeptides: LIM domain only protein? (SEQ ID No 35), LIM domain and actin-binding protein 1 (SEQ ID No 36), coatomer protein complex, subunit beta 2 (Beta prime), isoform CRA_b (SEQ ID No 37), ribonuclease inhibitor (SEQ ID No 38), PDZ and LIM domain protein 5 (SEQ ID No 39), reticulocalbin-1 (SEQ ID No 40), early endosome antigen 1 (SEQ ID No 41), septin-2 (SEQ ID No 42), actin-related protein 2/3 complex subunit 2 (SEQ ID No 43), septin 11 (SEQ ID No 44).

The inventors have unexpectedly realized that in order to optimize immuno-modulatory capacity the vesicle population may preferably have a greater abundance of serotransferrin than of annexin A2, and preferably greater abundance of annexin A2 than of connective tissue growth factor. Additional abundance patterns which are important for the MSCs and the extracellular vesicles immuno-modulatory capacity are also mentioned above.

In another aspect, the present invention pertains to a pharmaceutical composition comprising the MSC population and/or the extracellular vesicle population. In a further embodiment, the pharmaceutical composition may further comprise plasma of blood type AB, preferably at a concentration of at least around 1%, preferably around 10%. Furthermore, the MSCs are preferably present at a final concentration of between approximately 5×10⁵ and 4×10⁶ MSCs per ml of the pharmaceutical composition.

In yet another aspect, the present invention pertains to the MSC population and/or the vesicle population and/or the pharmaceutical composition for use in medicine.

In yet another aspect, the present invention pertains to the MSC population and/or the vesicle population and/or the pharmaceutical composition for use in the treatment of acute respiratory distress syndrome (ARDS), infant respiratory distress syndrome (IRDS), pulmonary hypertension (PH), or acute organ failure (for instance kidney, liver and/or heart failure) in connection with ARDS or IRDS.

In one embodiment, the MSC population and/or the vesicle population and/or the pharmaceutical composition may be used in the treatment of ARDS and/or acute organ failure in connection with ARDS through administration via a peripheral intravenous injection, central venous injection into the right atrium, injection into the right ventricle of the heart, and/or injection into the pulmonary trunk/artery

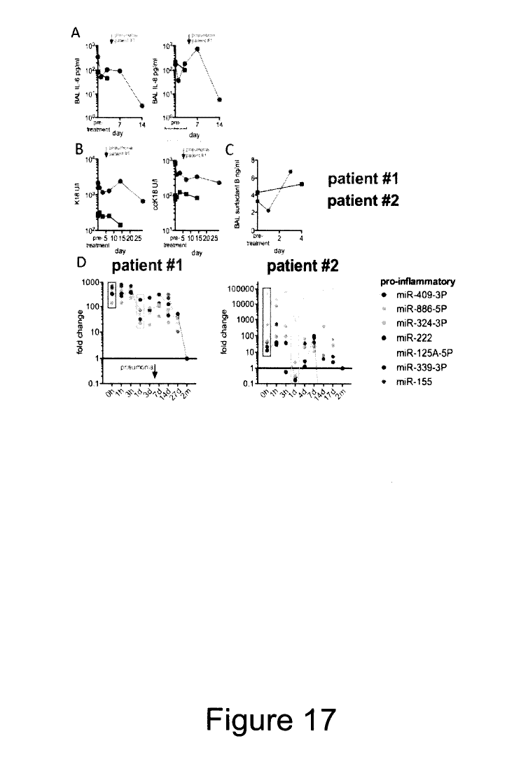

Patients that suffer from the above diseases/disorders and that are eligible for and/or are undergoing extra-corporal membranous oxygenation (ECMO) treatment are particularly suitable for treatment using the MSCs and/or the extracellular vesicles and/or the pharmaceutical compositions as per the present invention. Furthermore, one subgroup of patients where the therapeutic efficacy is optimal is the group of patients has having elevated levels of caspase-cleaved cytokeratin-18 (ccK18), as an indicator of epithelial apoptosis. Additionally, another subpopulation of patients is the patient group having elevated levels of at least one of the following microRNAs (miRs): miR-409-3P, miR-886-5P, miR-324-3P, miR-222, miR-125A-5P, miR-339-3P, and/or miR-155 (FIG. 17). In these patients the treatment has been shown to be particularly effective and the clinical outcomes have been excellent.

In a further aspect, the present invention relates to a method for culturing mesenchymal stem cells (MSCs). The culturing method does not utilize any non-human components and therefore greatly reduces the risk of eliciting immune responses, thereby possibly improving engraftment and therapeutic outcomes. In a first step, mesenchymal stem cells are obtained from a suitable source, followed by culturing the cells in a culture medium comprising lyzed human trombocytes (platelets), preferably at least 10⁷ (or even higher numbers, such as 10⁸ or 10⁹) lyzed human trombocytes per ml of culture medium.

MSCs may be obtained from various tissues, for instance bone marrow, blood, dermis, and/or the periosteum, using either allogeneic or autologous sources. When using an allogeneic source of MSCs it is of utmost importance to obtain cells from a young, healthy donor, who is preferably 35 years of age, preferably under 30 years of age, even more preferably under 25 years of age, and most preferably under 20 years of age.

In a further embodiment, when the MSCs are to be utilized for medical treatment of a patient suffering from any of the diseases and disorders that may be prevented, treated, cured or alleviated by MSCs, the cells and/or the extracellular vesicles may be harvested (and subsequently used for therapeutic treatment) when the population of MSCs have reached preferably at most 750×10⁶ cells, preferably at most 500×10⁶ cells, starting from at most 60 ml (cm³) of bone marrow aspirate. Thus, the entire procedure from obtaining the cells to the preparing a pharmaceutical composition may entail aspirating not more than 60 ml (preferably not more than 40 ml and even more preferably not more than 30 ml) from the bone marrow of a healthy donor (preferably from crista iliaca and/or sternum, expanding the MSCs obtained from the aspirate to preferably not more than 750*10⁶ cells, and, finally, harvesting the cells to prepare a pharmaceutical composition to be administered to a patient. The MSCs and/or the extracellular vesicles derived from the MSCs obtained by the methods of the present invention display a highly surprising level of therapeutic potency and a significantly smaller number of cells may hence be administered to a patient without reducing the therapeutic efficacy, thereby simplifying and speeding up the entire procedure. The key behind the therapeutic potency is the fact that the MSCs and/or the extracellular vesicles may be selected based on the immuno-modulatory criteria of the present invention, meaning that the MSCs and/or the extracellular components have a specific, advantageous proteomics profile and immuno-modulatory capacity. Another key aspect behind the enhanced therapeutic potency lies in the lower number of cell divisions the MSCs in accordance with the present invention have undergone, meaning that their beneficial immuno-modulating characteristics are conserved. When MSCs are obtained from the donor they may be frozen immediately, followed by thawing and starting of the cell culture upon the need for therapeutic intervention (alternatively, cells may be obtained (and cultivated directly) from a donor when a clinical need for MSCs arises). Surprisingly, it is crucial to not cultivate the MSCs for any longer period of time, preferably less than 5 passages (after obtaining the cells from the donor or after thawing), and even more preferably not more than 3 or 2 passages, in order to conserve the potency of the MSCs. The limited expansion of the bone marrow aspirate to not more than 750×10⁶ cells from not more than 60 ml (preferably not more than 50 ml, preferably not more than 40 ml, or even more preferably not more than 40 ml), and the limited number of passages, represent a completely novel approach in comparison to the existing art, with clearly beneficial therapeutic effects.

In yet another aspect, the present invention pertains to a cell culture composition for culturing MSCs. The cell culture composition comprises a cell culture medium (for instance Dulbecco's Modified Eagles Medium (DMEM), having a glucose concentration of between 0.5 and 2 g/l (preferably 1 g/l) (e.g. Gibco, InVitrogen Corp, #31885-023)), and lyzed human trombocytes (platelets), preferably at least 10⁷ lyzed platelets per ml of cell culture composition. The inclusion of lyzed human trombocytes is highly advantageous as it reduces the risk of immune responses to foreign antigens introduced when using conventional non-human serum (e.g. fetal calf serum). The platelet-containing culture medium/composition may be obtained using the following method: (1) isolating platelets from human blood (using conventional isolation techniques), (2) lyzing the platelets (preferably using radiation, e.g. ionizing radiation at around 24 Gy), (3) mixing the platelet lyzate and heparin, (4) mixing the platelet-heparin mixture with DMEM or any other suitable cell culture medium (the medium preferably containing suitable additives, such as antibiotics and/or antimycotics), and (5) centrifuging to eliminate any aggregates (normally at approximately 900 g for approximately 10 minutes).

The cell culture composition may comprise additional components, such as conventional antibiotic and antimycotic drugs (e.g. antibiotic/antimycotic, 100× Gibco, #15240-062), nutrients, or other additives. The DMEM normally comprises the standard ingredients of low glucose DMEM, namely amino acids, salts (normally calcium chloride, potassium chloride, magnesium sulfate, sodium chloride, and monosodium phosphate), glucose, iron, and vitamins (typically folic acid, nicotinamide, riboflavin, and vitamin B₁₂).

In a further aspect, the present invention relates to a pharmaceutical composition comprising between 5×10⁵ and (500,000) and 3×10⁶ (3,000,000) MSCs per ml in a pharmaceutically acceptable carrier. The MSCs in the pharmaceutical composition may advantageously be obtained by the methods according to the present invention but the MSCs may alternatively be obtained using other suitable methods.

In one embodiment, the pharmaceutical composition may further comprise plasma of blood type AB. Surprisingly, plasma of blood type AB may advantageously be used irrespective of the blood type of the patient to which the pharmaceutical composition is to be administered. Without wishing to be bound by any theory, it is surmised that the absence of antibodies against either A or B antigens is advantageous when administering the pharmaceutical composition to a patient.

The plasma (preferably of blood type AB) may be present at any concentration above 1%, preferably around 10%. The plasma may preferably be obtained fresh, normally cryo-reduced, and subsequently stored at −20° C. until use. In one embodiment, the pharmaceutically acceptable carrier may be an aqueous solution comprising at least 5% w/v sodium chloride, but other pharmaceutically and physiologically acceptable carriers may also be employed. The concentration of sodium chloride in the aqueous solution is preferably around 9% w/v.

In further embodiments, the MSCs and/or the extracellular components of the pharmaceutical composition may be from an allogeneic and/or an autologous cell source. If the cells of the pharmaceutical composition are obtained from an allogeneic donor it is crucial that the donor is a young, healthy individual under 30 years of age, preferably under 25 years of age, and even more preferably under 20 years of age.

In a further aspect, the present invention relates to a method for preparing a pharmaceutical composition comprising MSCs, comprising the following steps of culturing MSCs of crista iliaca and/or sternum origin in a cell culture composition comprising lyzed human trombocytes, passaging said MSCs not more than 5 times, and, re-suspending the MSCs so obtained in saline solution to a final concentration of between approximately 5×10⁵ and 4×10⁶ MSCs per ml.

The pharmaceutical composition may be obtained by aspirating between 20-60 ml (e.g. 20, 25, 30, 35, 40, 45, 50, 55, or 60 ml) of bone marrow from either sternum or Crista iliaca using an aspirate needle that is inserted through the skin using manual pressure until the needle hits the bone. When the needle hits the bone, with a twisting motion of the hand the needle is advanced through the bone cortex and into the marrow cavity. Once the needle is in the marrow cavity, a syringe is attached and used to aspirate liquid bone marrow. A twisting motion may preferably be performed during the aspiration to avoid excess content of blood in the sample, which might be the case if an excessively large sample from one single point is taken.

After aspiration, the cells are normally washed in a suitable liquid, for instance phosphate-buffered saline (PBS), and the washed cells may then be re-suspended in Dulbecco's modified Eagle's medium-low glucose supplemented with trombocyte (platelet) lysate and plated at a density of e.g. 160 000 cells per cm². Cultures are maintained at 37° C. in a humidified atmosphere containing 5% CO₂, preferably in 175 cm² flasks. When the cultures are near confluence (>80%), the cells may be detached by treatment with trypsin and EDTA and replated at a density of 4000 cells per cm². When approximately 2×10⁶ cells or more are obtained, the cells are harvested and either cryopreserved in 10% dimethyl sulphoxide (DMSO) or washed repeatedly with PBS and re-suspended to a final concentration of between approximately 5×10⁵ and 4×10⁶ cells per ml in saline solution. The saline solution into which the MSCs are re-suspended may preferably comprise blood plasma, preferably plasma of blood type AB.

After aspiration of the MSCs from the bone marrow of a donor, it is crucial to not cultivate the MSCs for any longer period of time, preferably less than 5 passages (after obtaining the cells from the donor or after thawing), and even more preferably not more than 3 or 2 passages, in order to conserve the potency of the MSCs

Various criteria for release of MSCs for clinical use need to be met, including the absence of visible clumps and the absence of contamination by pathogens (as documented by aerobic and anaerobic cultures before release), the MSCs shall preferably display a spindle-shape morphology, have a viability that preferably is greater than 95%, and immune phenotyping shall preferably show expression of CD73, CD90, and CD105 surface molecules (>90%) and absence of CD34, CD45, CD14, and CD3.

In yet additional embodiments, the pharmaceutical composition as per the present invention may further comprise extracellular vesicles (for instance exosomes) obtainable from the MSCs. Exosomes may be added to the pharmaceutical composition from a separate culture of MSCs or they may be derived from the MSCs that are themselves to be included in the pharmaceutical composition.

To optimize the properties of the pharmaceutical composition and to avoid coagulation upon injection, the pharmaceutical composition may comprise heparin or any other anticoagulation factor.

The present invention, in a further aspect, thus relates to the use of the pharmaceutical compositions according to the present invention for use in medicine, and specifically in the treatment of diseases and disorders such as acute respiratory distress syndrome (ARDS), infant respiratory distress syndrome (IRDS), pulmonary hypertension (PH), congenital heart diseases, and acute organ failure (optionally in connection with ARDS and/or IRDS), for instance heart failure, kidney failure, and/or liver failure.

Acute respiratory distress syndrome (ARDS) is an important cause of acute respiratory failure and is often associated with multiple organ failure. Several clinical disorders can precipitate ARDS, for instance viral and/or bacterial pneumonia, aspiration of gastric contents, sepsis, surgery, and major trauma. The clinical criteria for ARDS are normally the following:
Acute onset: 20-50% of acute lung injury patients will develop ARDS within 7 days.
Capillary wedge pressure (PCWP) 518 mmHg or no evidence of cardiac failure. Chest X-ray shows bilateral infiltrates.
Refractory hypoxaemia: ARDS is present when PaO₂:FiO₂<200.
Physiologically, ARDS is characterized by increased permeability pulmonary edema, severe arterial hypoxemia, and impaired carbon dioxide excretion and is the result of an on-going inflammatory response.
Clinically, up-regulation of inflammatory cytokines frequently persists in the patients.

Infant respiratory distress syndrome (IRDS) of the newborn is the most common cause of respiratory distress in premature infants, correlating with structural and functional lung immaturity. The pathophysiology is characterized by an ongoing inflammatory response giving immature type II alveolar cells that produce less surfactant, causing an increase in alveolar surface tension and a decrease in compliance. The resultant atelectasis causes pulmonary vascular constriction, hypoperfusion, and lung tissue ischemia. Hyaline membranes form through the combination of sloughed epithelium, protein, and edema. Persistent respiratory distress syndrome leads to bronchopulmonary dysplasia, characterized by typical chest radiography findings and chronic oxygen dependence.

Pulmonary hypertension (PH) is an increase in blood pressure in the pulmonary artery, pulmonary vein, and/or the pulmonary capillaries and it can be a severe disease with a markedly decreased exercise tolerance and heart failure. Evidence is accumulating to suggest that inflammation plays a significant role in the pathogenesis of PH. Endothelial cells play an important role in inflammation and immune reactions, and inflammatory cytokines cause endothelial dysfunction. Endothelial dysfunction is a hallmark of PH, consisting in reduced availability of vasodilators and antiproliferative factors and increased production of vasoconstrictors and vascular proliferative factors. Up-regulation of inflammatory cytokines and perivascular inflammatory cell infiltration have been detected in the lungs of patients with PH. Persistent PH of the newborn occurs when pulmonary vascular resistance fails to decrease soon after birth as with normal transition. The etiology may be idiopathic or secondary to meconium aspiration syndrome, pneumonia or sepsis, respiratory distress syndrome, or transient tachypnea of the newborn. The increased pulmonary hypertension gives rise to an ongoing inflammatory response in the lung.

Congenital heart disorders giving rise to cyanotic heart disease includes transposition of the great arteries and Tetralogy of Fallot (TOF). Noncyanotic heart lesions that cause a pulmonary overflow state leading to congestive heart failure. These lesions include large septal defects, patent ductus arteriosus, and coarctation of the aorta. The increased hypertension as well as cyanosis gives rise to an inflammatory response in the lung.

Thus, the common denominator between the various diseases and disorders in accordance with the present invention is the inflammatory aspect, either in (1) the pulmonary system as such, (2) emanating from the pulmonary system and involving other parts of the body such as the kidney (e.g. kidney or liver failure), or (3) emanating from some other body part and involving the pulmonary system (e.g. as in congenital heart disorders).

The present inventors have unexpectedly discovered that the administration route of the MSCs is important to achieve therapeutic efficacy. For instance, in the treatment of acute respiratory distress syndrome (ARDS), a pharmaceutical composition comprising MSCs and/or extracellular vesicles derived from said MSCs may preferably be administered via peripheral intravenous injection, central venous injection into the right atrium, injection into the right ventricle of the heart, and/or injection into the pulmonary trunk/artery. Specifically, the pharmaceutical compositions as per the present invention display considerable therapeutic efficacy in the patient group that (1) suffer from ARDS, IRDS, PH, or any other pulmonary disease or disorder within the scope of the present invention, and (2) are eligible and/or are undergoing extra-corporal membranous oxygenation (ECMO) treatment. The pharmaceutical compositions in accordance with the present invention can thus be administered both to patients that have already been placed on ECMO support, and to patients that are eligible but have not yet commenced ECMO treatment. Furthermore, as above-mentioned, subjects having certain miRNA profiles are particularly eligible for treatment. More specifically, patients having upregulated levels of at least one of the following microRNAs (miRs) respond to treatment in a surprisingly rapid and stable manner: miR-409-3P, miR-886-5P, miR-324-3P, miR-222, miR-125A-5P, miR-339-3P, and/or miR-155 (FIG. 17). Furthermore, another subgroup of patients where the therapeutic efficacy is optimal is when the subject has elevated levels of caspase-cleaved cytokeratin-18 (ccK18), as an indicator of epithelial apoptosis.

In one embodiment, in patients that are placed on ECMO support, the administration mode and route of the pharmaceutical compositions may be optimized by reducing the forward flow of the ECMO to zero, either by turning off the machine and/or by clamping a suitable part of the machine to stop the flow. By reducing the flow rate to zero for a time sufficient to permit administration of the pharmaceutical composition comprising the MSCs and/or the extracellular vesicles obtainable from the MSCs the engraftment and therapeutic efficacy may be enhanced.

In a further aspect, the present invention relates to a method of preventing, treating, and/or alleviating diseases and/or disorders selected from the group comprising ARDS, IRDS, acute organ (e.g. kidney, heart, and/or liver) failure (optionally in connection with IRDS and/or ARDS), and congenital heart diseases (with pulmonary involvement). The treatment methods may comprise the steps of providing a therapeutic dose of MSCs and/or extracellular vesicles obtainable from said MSCs, and administering said therapeutic dose to a patient in need thereof. In one embodiment, the therapeutic dose of MSCs and/or exosomes obtainable from said MSCs comprises at least 500,000 MSCs per kg of body weight, preferably at least 1,000,000 MSCs per kg of body weight, or even more preferably at least 1,500,000 MSCs per kg of body weight.

### EXPERIMENTS AND TRIALS

### Materials and Methods

Peripheral blood mononuclear cells (PBMCs) used in in vitro studies were retrieved from patient and from healthy donors, isolated by density gradient-based centrifugation, and stored in 10% DMSO in liquid nitrogen until further analysis. For further purification of T-cells, a paramagnetic bead-based selection was utilized (Miltenyi Biotec, Bergisch Gladbach Germany). Bone marrow-derived MSCs (BM-MSCs) were co-cultured with allogeneic T-cells (MSC:T-cell ratio 1:5, 1:10) for 5 days. T-cells were stimulated with activating anti-CD2/CD3/CD28 antibodies (Miltenyi Biotec) in a 0.5 bead per cell ratio. PBMCs were cultured for five days in the presence of MSCs (MSC:PBMC ratio 1:5 and 1:10) and the monocytic compartment was subsequently analyzed.

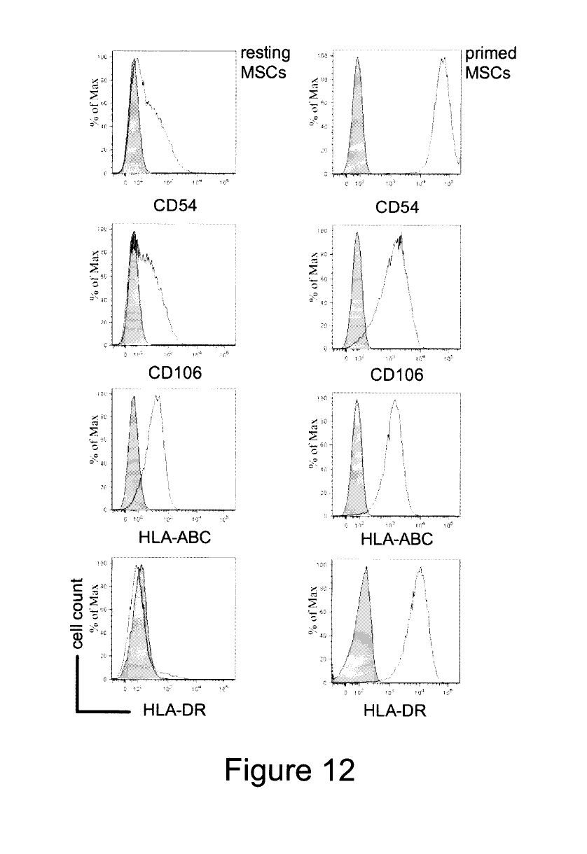

Polymorphonuclear leukocytes (PMNs) from buffy coats of healthy donors were ultrapurified under endotoxin-free conditions, as previously described. PMNs (>95% purity) and MSCs were co-cultured for up to 40 h in presence or absence of 100 ng/ml of LPS. In all cases, MSC were plated 72 hours before the start of co-cultures. In selected experiments, MSCs were pre-treated with recombinant human IFN-γ (10 ng/ml) and TNF-α (15 ng/ml) for 48 h (FIG. 12), gently washed twice with fresh medium to remove residual cytokines, and finally co-cultured with freshly isolated PMNs. MSC and PMN cocultures were stained with May Grunwald-Giemsa dye to observe cell morphology following reciprocal interaction.

BAL Fluid and Serum Analyses

Levels of pro- and anti-inflammatory cytokines in serial samples of BAL fluid and serum were assessed using a multiplex cytokine assay (Millipore, Mass., US) on a Luminex machine (Luminex, Millipore). In BAL fluid, surfactant protein B concentration was determined by enzyme-linked immunosorbant assay (ELISA) (Uscn Life Science Inc, Houston, US). Caspase-cleaved K18 (ccK18) and total K18 were measured using M30-Apoptosense® ELISA (Peviva AB, Bromma, Sweden) and M65 EpiDeath® ELISA (Peviva AB), respectively.

miRNA Analysis

The EV purification and miRNA isolation was performed by Exosome Diagnostic Inc. (NY, US). In brief, 1.5-2 ml of blood sample from each time point was purified using the EXO50 (Exosome Diagnostics, Martinsried, Germany). The eluted RNA was processed for microRNA analysis using the Low Sample Input protocol for the TaqMan® OpenArray® Human MicroRNA Panel (Life Technologies, US). Megaplex™ RT Primers, Human Pool A and Human Pool B were used for reverse transcription followed by a pre-amplification step according to manufacturer's protocol. The pre-amplified material was diluted 1:20 in TE (pH 8.0) and analyzed on the TaqMan OpenArray Human MicroRNA Panel for final qPCR detection. In total 758 miRNAs were assayed and 200 to 300 miRNAs gave a detectable CT value for each time point.

U6 was used to normalize for sub-array-to-sub-array variation and for differences between two Megaplex pools, because this RNA was present in every sample and generated Ct values in a reliable range between 12 and 22. Delta Ct values were calculated for each miRNA by subtracting the U6 Ct value located on the same sub-array. Next, we corrected for variation of the total RNA amount. We therefore subtracted each Ct by the median Ct value of 128 miRNAs present in all samples in the first patient and by the median Ct of 79 miRNAs present in all samples in the second patient. This procedure was performed for all samples. Missing values were entered with the highest observed normalized Ct value per patient. Finally, the fold change for every miRNA relative to the normal healthy control sample was calculated.

Expansion of Mesenchymal Stem Cells

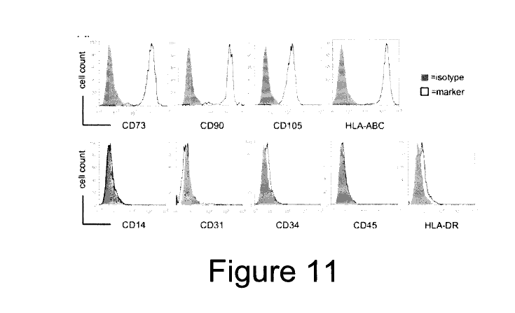

Twenty-eight ml of bone marrow was aspirated from a 49 year old HLA-mismatched third party healthy male volunteer. Clinical-grade MSCs were generated under good manufacturing practice (GMP) conditions according to a common protocol elaborated by the EBMT Developmental Committee, accredited by the Swedish National Board of Health and Welfare. Bone marrow mononuclear cells (146×10⁶) were seeded into 175 cm² flasks (Falcon, Franklin Lakes, N.J., USA) in Dulbecco's modified Eagles Medium-Low Glucose (DMEM-LG, Life Technologies, Gaithersburg, Md., USA) supplemented with lysed human platelets (final concentrations ranging from 10⁷ to 10⁹, normally 10⁸/mL). When the cultures were near confluence (>80%), the cells were detached by treatment with trypsin and EDTA (Invitrogen, Grand Island, N.Y., USA) and re-plated once at a density of 4,000 cells/cm². MSCs were harvested and cryopreserved in 10% Dimethyl Sulfoxide (WAK-Chemie Medical GmbH, Germany). After thawing, the cells were washed three times in PBS and re-suspended in 0.9% saline solution with the addition of 10% AB plasma, to a final concentration of 2×10⁶ cells/ml. MSC release criteria for clinical use included: absence of visible clumps, spindle shape morphology, absence of contamination by pathogens (bacteria and mycoplasma) and viability >95%. MSCs expressed CD73, CD90, CD105, HLA-ABC and were negative for CD14, CD31, CD34, CD45 and HLA-DR (FIG. 11).

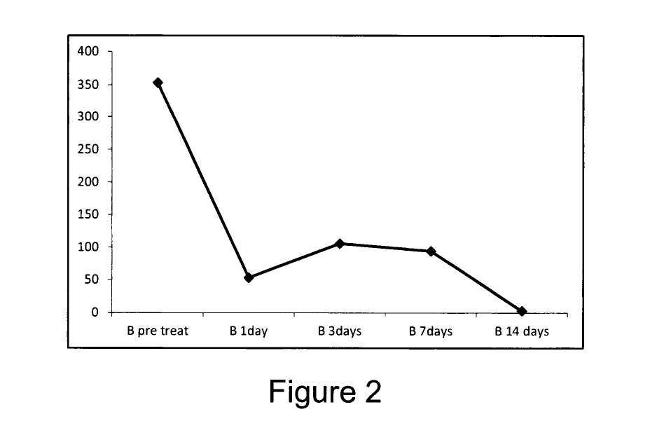

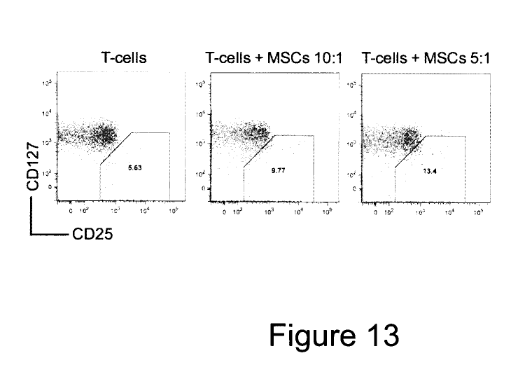

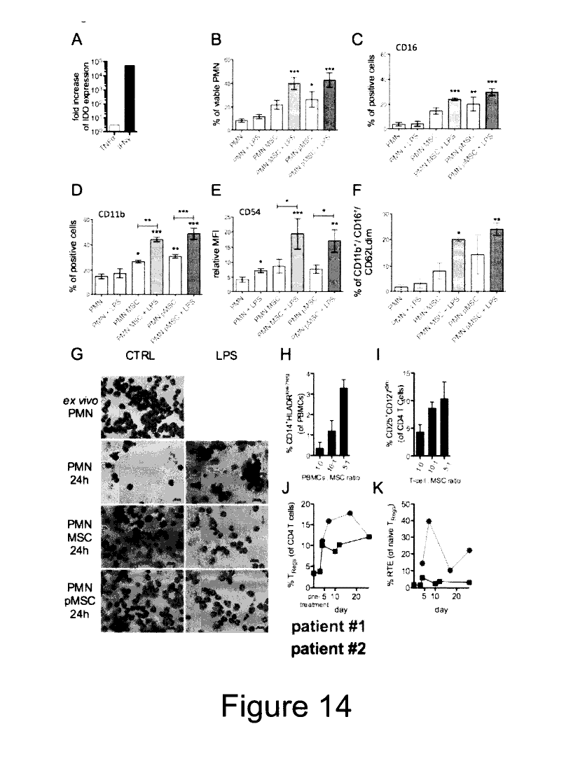

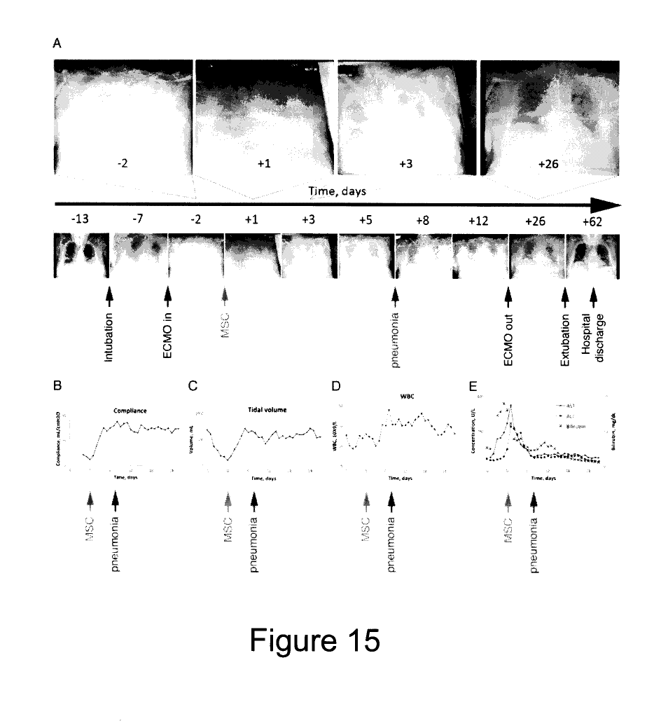

Assessment of Immuno-Modulatory Capacity
Ex-vivo expanded MSCs were pre-treated (primed MSCs, pMSCs) or not (MSCs) with 10 ng/ml of IFN-γ and 15 ng/ml of TNF-α for 48 hours before used in co-cultures with PMNs with or without activation by endotoxin (100 ng/ml of lipopolysaccharides (LPS)). Following inflammatory priming, the MSCs up-regulated cell surface expression of CD54 (ICAM-1), CD106 (VCAM-1) and HLA-ABC and -DR (FIG. 2), as well as the expression of indoleamine 2,3-dioxygenase (IDO), a potent mediator of many MSC immune regulatory functions (FIGS. 12, 13 and 14).
Fold increase of IDO expression after TNFα stimulation of donor cell MSCs should be <10 and fold increase of IDO expression after stimulation of INFy should be more than >100 (p0,05). MSCs was cultured in the presence of TNF-α (15 ng/mL) or IFN-γ (10 ng/mL).
Viability of PMN should be significantly improved (p0.05), preferably improved by at least 10%, or more preferably by at least 20%, after adding in either MSCs and LPS or pMSC with or without LPS as a co-culture. Viability and expression levels of surface markers by control or LPS-stimulated PMNs were investigated after 40 hours of direct co-culture with either resting or pMSCs. CD16 (FcγR-III) expression was used as surrogate marker of PMN viability. In the absence of LPS, PMN survival was enhanced only in presence of pMSCs. When LPS was added to the co-culture, both resting and pMSCs protected PMNs from apoptosis. Accordingly, the percentage of CD16-positive PMNs matched with the percentage of viable PMNs in all culture conditions. In parallel, expression of CD11b and CD54 is typically associated with PMN activation status. As expected, the percentage of CD11b-positive PMNs was higher in the presence of MSCs and further enhanced by LPS treatment, while CD11b relative mean fluorescence intensity (rMFI) did not change significantly, suggesting that more PMNs are becoming activated following MSC or pMSC exposure. Meaning that CD11b should be significantly improved (p 0,05) in pressens of MSCs, pMSCs with or without LPS. In contrast, CD54 rMFI should be significantly (p0,05) up-regulated by LPS treatment and this effect was enhanced by co-culture with MSCs. Overall, the higher PMN survival and activation triggered by MSCs suggests that MSCs may influence the PMN-dependent innate response through functional modifications rather than pro-apoptotic effects (FIG. 14).
Donor MSCs should preferably increase the number of myeloid-derived suppressor cells (MDSCs) by a factor 1.5, or more preferably a factor 2. Co-culturing PMNs with MSCs led to a marked increase in mature CD11b^{bright}/CD16^{bright}/CD62L^{dim} (N2-type) cells with hypersegmented nuclei consistent with granulocytic MDSCs (FIG. 14). Similarly, co-culturing MSCs with healthy control human peripheral blood mononuclear cells (PBMCs) at different ratios promoted a significant increase (p0,05) CD14⁺HLA-DR^{low} monocytes resembling monocytic MDSCs (FIG. 14).
Donor MSCs should significantly increase (p 0,05), preferably 1.5-fold or even more preferably 2-fold, the number of regulatory T-cells as exemplified by CD4⁺CD25^{high}CD127^{low} regulatory T-cells (T_{Regs}), a key immune regulatory cell population, was also expanded in co-culture experiments with PBMCs (FIGS. 13 and 14). This corresponded with increased levels of circulating CD4⁺CD25^{high}CD127^{low} T_{Regs} that was observed in observed in treated patients' peripheral blood for up to 20 days following MSC administration (T_{Regs} among CD4⁺ T-cells in healthy controls (n=11) 3.84+/−1.60%, T_{Reg} range in patients 3.34-17.8%) (FIG. 14). This finding could have resulted from an elevated thymic output as indicated by the increased proportion of CD31⁺ recent thymic emigrants among CD45RA⁺ naive T_{Regs} in patients and/or the MSC-stimulated conversion of conventional T-cells in the periphery (FIG. 14).

Male Patient with Virus-Induced ARDS

Patient 1: A 58-year-old man with history of hypertension was hospitalized eight days after onset of high fever, cough, and dyspnea. Chest X-ray (CXR) demonstrated diffuse bilateral infiltrates and polymerase chain reaction (RT-PCR) of bronchoalveolar lavage (BAL) fluid was positive for influenza A H1N1. Oseltamivir (Tamiflu, Roche, Switzerland) was initiated but the patient deteriorated over the next two days with increased bilateral opacities on CXR and progressive respiratory failure requiring mechanical ventilation, followed by initial veno-venous (vv)- and later veno-arterial (va)-ECMO (CardioHelp, Maquet, Germany) for refractory hypoxemia. Acute kidney failure developed requiring continuous renal replacement therapy (CRRT).

Over the next four days, the patient's condition worsened with persistent hypoxemia, progressive bilateral infiltrates, and liver failure. At this juncture, following extensive discussions with the family, the hospital administration, and ethics board, HLA mismatched allogeneic bone marrow-derived MSCs obtained from a healthy volunteer were systemically infused through a central venous catheter positioned in the right atrium of the heart. The total dose (approximately 2×10⁶ cells/kg recipient weight) was divided into aliquots of 5 mL of cell suspension given at six time points over 20 minutes. To avoid infusion of MSCs into the ECMO circuit, the ECMO outflow cannula was clamped during each infusion, resulting in infusion inot the right atrium of the heart. At the time of infusion, the patient had no detectable influenza A H1N1 particles, as assessed by RT-PCR and had no other detectable active infection.

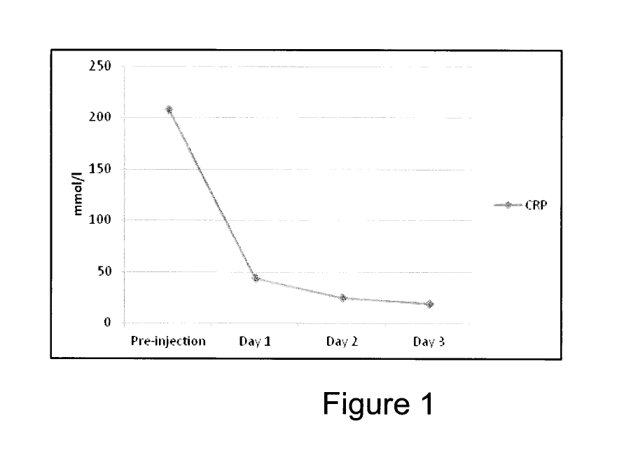

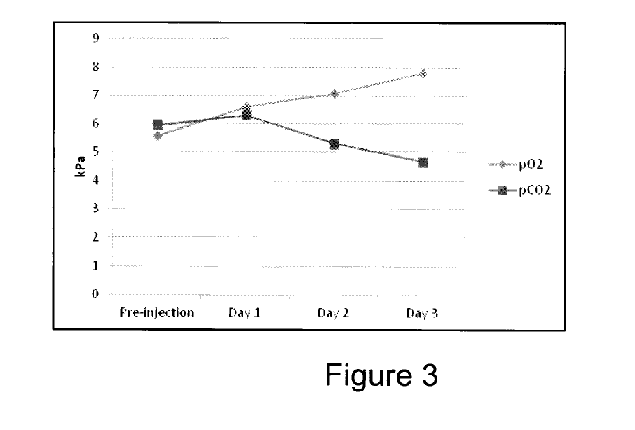

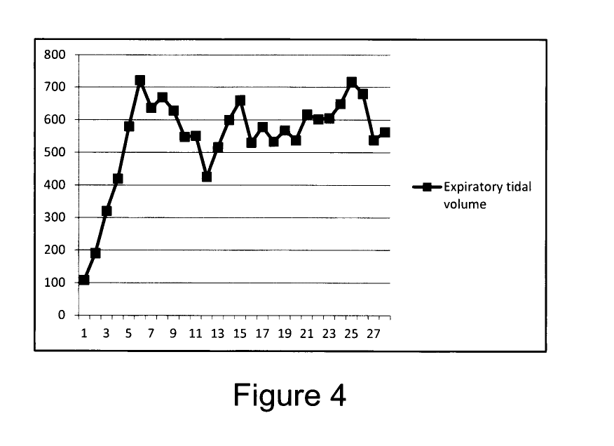

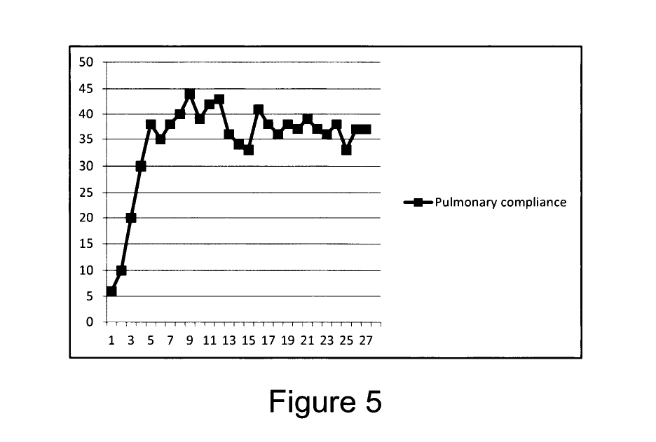

Within 24 hours clear signs of improvement are evident, both in terms of clinical chemistry parameters and pulmonary symptoms. CRP decreased strongly after administration of the MSCs (FIG. 1), and so did various inflammatory cytokines, for instance the bronchoalveolar lavage (BAL) IL-6 (FIG. 2), showing decreasing inflammatory response in the lung post-treatment. The partial oxygen pressure pO₂ increased significantly from pre-injection to day 3 post-injection, and the pCO₂ displayed a concomitant decrease (FIG. 3). Additionally, tidal volume (FIG. 4) and compliance (FIG. 5) increased significantly in response to the MSC treatment.

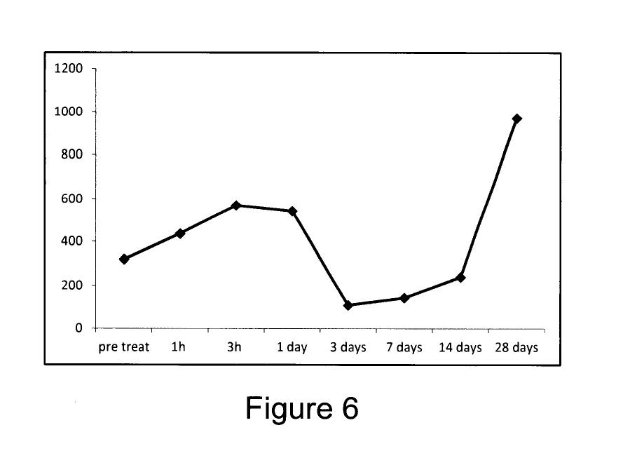

Finally, increased serum levels of RANTES approximately four weeks after treatment is indicative of stem cell mobilization in response to the MSC injection (FIG. 6).

Kidney Failure in Patient with Virus-Induced ARDS

A male patient presenting with virus-induced pneumonia develops subsequent ARDS, which in turn induces acute kidney failure requiring dialysis.

The patient is eligible for ECMO treatment but prior to commencing treatment a pharmaceutical composition comprising around 10⁶ MSCs per kg of body weight is administered to the patient (via peripheral venous injection), instead of placing the patient on ECMO support.

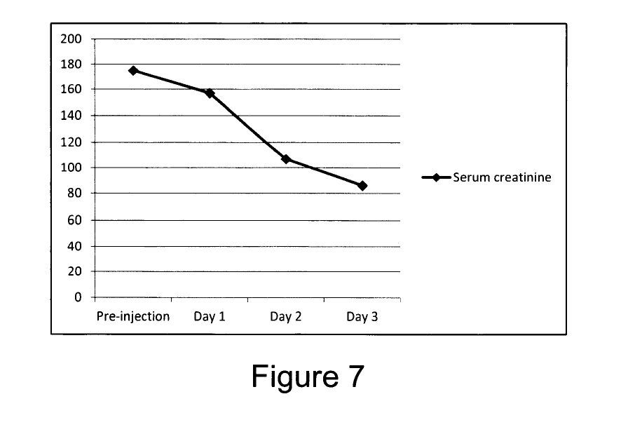

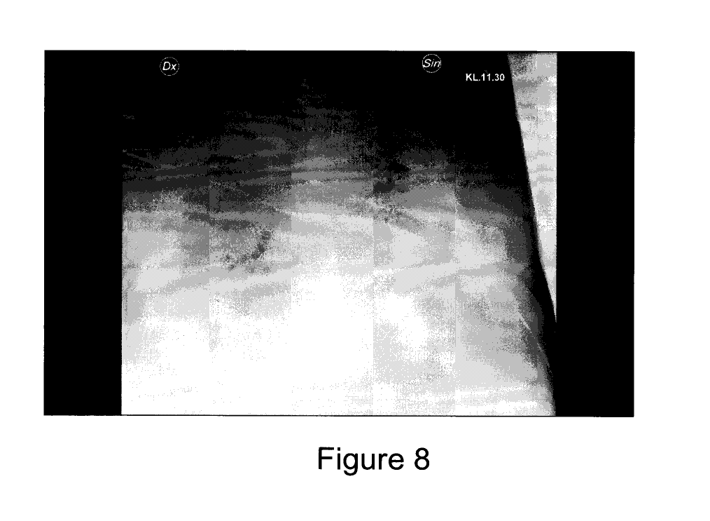

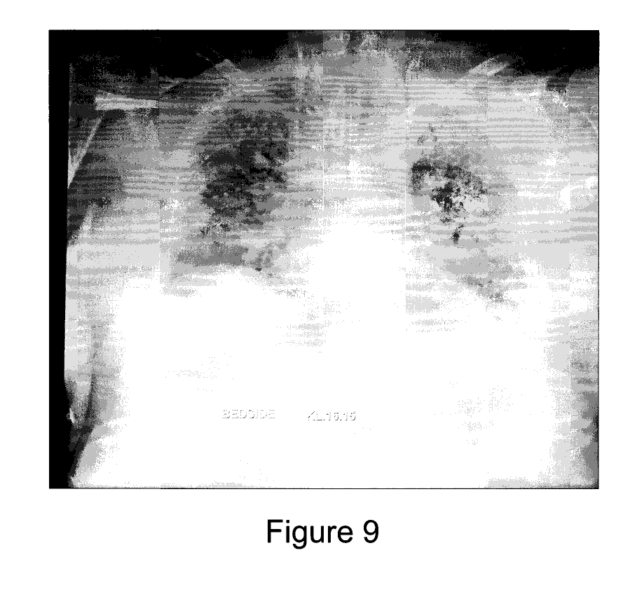

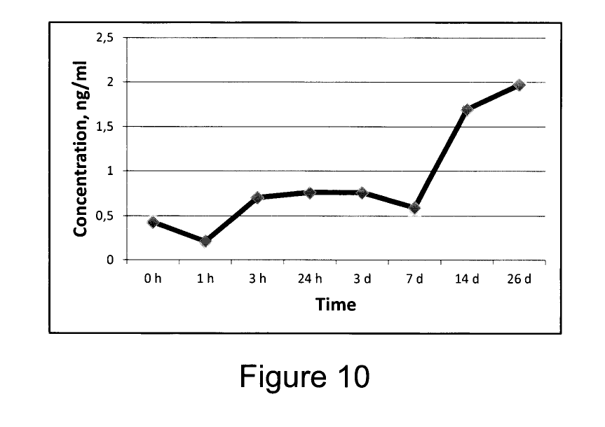

Post-injection of the MSCs the kidney failure resolved, with serum creatinine levels decreasing considerably (FIG. 7) and the pulmonary symptoms were eliminated, as evidenced by lung X-ray (FIGS. 8,9, and 15) and recovering plasma surfactant B levels (FIG. 10), with the patient subsequently being removed from ECMO treatment and dialysis.

Treatment of Infection-Induced ARDS and Transfusion-Related Acute Lung Injury (TRALI)

A 40-year-old man with no previous medical history was admitted to the hospital due to malaise, weight loss, night sweats, gingival hyperplasia and fever. Acute myeloid leukemia was diagnosed in the bone-marrow aspirate and induction chemotherapy was initiated. Due to caries and dental root abscesses, several teeth were extracted at the time of initiation of cytotoxic therapy. The following day, the patient became progressively hypoxemic and developed acute respiratory failure requiring intubation and mechanical ventilation. Blood and sputum cultures were negative, while CXR demonstrated bilateral infiltrates.

Despite treatment with broad spectrum antibiotics in the now neutropenic patient, progressive hypercapnia and hypoxia developed over the next two days and the patient was placed on vv-ECMO. Echocardiography demonstrated normal cardiac function. The patient was initially stable on ECMO but the clinical course was complicated by severe transfusion-dependent cytotoxic chemotherapy-induced thrombocytopenia.

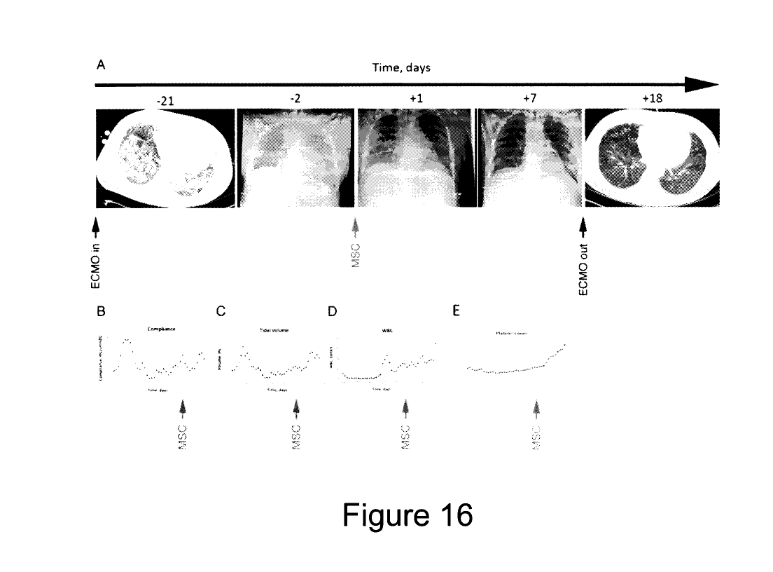

Given the large fluid volume from transfusions, CRRT was initiated in order to maintain appropriate fluid balance and reduce interstitial oedema. Fourteen days after initiation of ECMO the patient continued to deteriorate with increasing hypoxia and progressive bilateral infiltrates on CXR. Possible contributing factors included progressive ARDS, transfusion-related acute lung injury (TRALI), and/or undiagnosed infection although blood and sputum cultures remained negative. Following extensive discussion with the family, the hospital administration, and ethics board, HLA mismatched allogeneic bone marrow-derived MSCs (2×10⁶ cells/kg recipient weight) were similarly administered as in the first patient twenty-eight days after initiation of ECMO. As can be seen from FIG. 16, there was progressive decrease in pulmonary infiltrates starting the first day after MSC infusion. By day 7, the CXR improved back to normal and the patient was weaned off ECMO the following day and extubation at day 12. Pulmonary compliance and tidal volumes improved back to normal levels during the first week. After MSC infusion, the bone-marrow function continued to be reconstituted with resolution of leukopenia and thrombocytopenia.

1. A method for obtaining immuno-modulatory MSCs, comprising the steps of:
(i) culturing MSCs of a suitable origin;
(ii) establishing that the MSC population fulfil at least one of the following immuno-modulation criteria:
(a) the MSC population is positive for at least one of the following polypeptides: vimentin (SEQ ID No 1), caldesmon (SEQ ID No 2), annexin A1 (SEQ ID No 3), 14-3-3 protein epsilon (SEQ ID No 4), ADP ribosylation factor 1 (SEQ ID No 5), calnexin (SEQ ID No 6), ADP ribosylation factor 5 (SEQ ID No 7), transforming protein RhoA (SEQ ID No 8), CD44 (SEQ ID No 9), coactosin-like protein (SEQ ID No 10), mitogen-activated protein kinase 3 (SEQ ID No 11), insulin-like growth factor-binding protein 7 (SEQ ID No 12), N-acetyl-glucosamine-6-sulfatase (SEQ ID No 13), cellular retinoic acid-binding protein 2 (SEQ ID No 14), transcription elongation factor B polypeptide 1 (SEQ ID No 15), NEDD8 (SEQ ID No 16), fatty acid-binding protein, heart (SEQ ID No 17);
(b) a population of extracellular vesicles derived from the MSC population is positive for at least one of the following polypeptides: serotransferrin (SEQ ID No 18), versican core protein (SEQ ID No 19), annexin A2 (SEQ ID No 20), serine protease HTRA1 (SEQ ID No 21), insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerise (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34);
(c) the MSC population in (a) displays the following order of polypeptide abundance: vimentin>Annexin A1;
(d) the extracellular vesicle population in (b) displays the following order of polypeptide abundance: serotransferrin>annexin A2;
(e) the fold-increase expression of indoleamine 2,3-dioxygenase (IDO) in the MSC population is <10 when the MSCs are primed with 15 ng/mL TNF-alpha;
(f) the fold-increase expression of indoleamine 2,3-dioxygenase (IDO) in the MSC population is >100 when the MSCs are primed with 10 ng/mL IFN-gamma;
(g) the viability of polymorphonuclear neutrophils (PMNs) is increased by at least 20% when co-cultured with MSCs from the MSC population primed with IFN-gamma or TNF-alpha in accordance with (e) or (f);
(h) the number of CD14⁺HLA-DR^{low} monocytes is increased at least 1.5-fold when healthy control human peripheral blood mononuclear cells (PBMCs) are co-cultured with the MSC population primed with IFN-gamma or TNF-alpha in accordance with (e) or (f);
(i) the number of CD4⁺CD25^{high}CD127^{low} regulatory T-cells (T_{Regs}) is increased at least 15-fold when healthy control human peripheral blood mononuclear cells (PBMCs) are co-cultured with the MSC population primed with IFN-gamma or TNF-alpha in accordance with (e) or (f);

2. The method of claim 1, wherein the MSCs fulfil at least criteria (e) and (f).

3. The method of claim 1, wherein the MSCs are passaged at most 5 times before clinical use.

4. A population of immuno-modulatory MSCs obtainable by the method of claim 1.

5. A population of immuno-modulatory MSCs having the following antigen profile: CD73+, CD90+, CD105+, CD34−, CD45−, CD14−, and CD3−.

6. The MSC population of claim 5, wherein the MSC population is positive for vimentin and/or Annexin A1.

7. The MSC population of claim 5, wherein the MSC population expresses a larger abundance of vimentin than of CD44.

8. The MSC population of claim 5, wherein the MSC population fulfils at least one of the following immuno-modulation criteria:
(j) the MSC population is positive for at least one of the following polypeptides: vimentin (SEQ ID No 1), caldesmon (SEQ ID No 2), annexin A1 (SEQ ID No 3), 14-3-3 protein epsilon (SEQ ID No 4), ADP ribosylation factor 1 (SEQ ID No 5), calnexin (SEQ ID No 6), ADP ribosylation factor 5 (SEQ ID No 7), transforming protein RhoA (SEQ ID No 8), CD44 (SEQ ID No 9), coactosin-like protein (SEQ ID No 10), mitogen-activated protein kinase 3 (SEQ ID No 11), insulin-like growth factor-binding protein 7 (SEQ ID No 12), N-acetyl-glucosamine-6-sulfatase (SEQ ID No 13), cellular retinoic acid-binding protein 2 (SEQ ID No 14), transcription elongation factor B polypeptide 1 (SEQ ID No 15), NEDD8 (SEQ ID No 16), fatty acid-binding protein, heart (SEQ ID No 17);
(k) a population of extracellular vesicles derived from the MSC population is positive for at least one of the following polypeptides: serotransferrin (SEQ ID No 18), versican core protein (SEQ ID No 19), annexin A2 (SEQ ID No 20), serine protease HTRA1 (SEQ ID No 21),
insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerise (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34);
(l) the MSC population in (a) displays the following order of polypeptide abundance: vimentin>annexin A1;
(m) the extracellular vesicle population in (b) displays the following order of polypeptide abundance: serotransferrin>annexin A2;
(n) the fold-increase expression of indoleamine 2,3-dioxygenase (IDO) in the MSC population is <10 when the MSCs are primed with 15 ng/ml TNF-alpha;
(o) the fold-increase expression of indoleamine 2,3-dioxygenase (IDO) in the MSC population is >100 when the MSCs are primed with 10 ng/ml IFN-gamma;
(p) the viability of polymorphonuclear neutrophils (PMNs) is increased by at least 20% when co-cultured with MSCs from the MSC population primed with IFN-gamma or TNF-alpha in accordance with (e) or (f);
(q) the number of CD14+HLA-DRlow monocytes is increased at least 1.5-fold when healthy control human peripheral blood mononuclear cells (PBMCs) are co-cultured with the MSC population primed with IFN-gamma or TNF-alpha in accordance with (e) or (f);
(r) the number of CD4+CD25high CD127low regulatory T-cells (TRegs) is increased at least 1.5-fold when healthy control human peripheral blood mononuclear cells (PBMCs) are co-cultured with the MSC population primed with IFN-gamma or TNF-alpha in accordance with (e) or (f);

9. A vesicle population comprising extracellular vesicles derived from the MSC population of claim 5.

10. The vesicle population of claim 9, wherein the extracellular vesicles are exosomes.

11. The vesicle population of claim 9, wherein the vesicle population is positive for at least one of the following polypeptides: serotransferrin (SEQ ID No 18), versican core protein (SEQ ID No 19), annexin A2 (SEQ ID No 20), serine protease HTRA1 (SEQ ID No 21), insulin-like growth factor-binding protein 3 (SEQ ID No 22), connective tissue growth factor (SEQ ID No 23), vinculin (SEQ ID No 24), neuroblast differentiation associated protein AHNAK (SEQ ID No 25), microtubule-associated protein 1B (SEQ ID No 26), fatty acid-synthase (SEQ ID No 27), triosephosphate isomerise (SEQ ID No 28), ATP-citrate synthase (SEQ ID No 29), calreticulin (SEQ ID No 30), vigilin (SEQ ID No 31), DNA-dependent protein kinase catalytic subunit (SEQ ID No 32), Rab GDP dissociation inhibitor beta (SEQ ID No 33), ATP synthase subunit beta, mitochondrial (SEQ ID No 34).

12. The vesicle population of claim 9, wherein the vesicle population displays the following order of polypeptide abundance: serotransferrin>annexin A2.

13. A pharmaceutical composition comprising the MSC population of claim 5.

14. The pharmaceutical composition claim 13, further comprising plasma of blood type AB at a concentration of at least 1%, preferably 10%.

15. A method of treatment comprising administering the MSC population of claim 5 to a subject in need thereof.

16. The method of claim 15, wherein the treatment is treatment of acute respiratory distress syndrome (ARDS), infant respiratory distress syndrome (IRDS), pulmonary hypertension (PH), or acute organ failure in connection with ARDS or IRDS.

17. The method of claim 15, wherein the treatment is treatment of ARDS and/or acute organ failure in connection with ARDS, wherein the MSC population is administered via peripheral intravenous injection, central venous injection into the right atrium, injection into the right ventricle of the heart, and/or injection into the pulmonary trunk/artery.

18. The method of claim 15, wherein the treatment is treatment of ARDS, wherein the patient suffering from ARDS is eligible for and/or is undergoing extra-corporal membranous oxygenation (ECMO) treatment.

19. The method of claim 15, wherein the treatment is treatment of ARDS and/or acute organ failure in connection with ARDS, wherein the subject has elevated levels of caspase-cleaved cytokeratin-18 (ccK 18) as an indicator of epithelial apoptosis.

20. The method of claim 15, wherein the treatment is treatment of ARDS and/or acute organ failure in connection with ARDS, wherein the subject to be treated has elevated levels of at least one of the following microRNAs (miRs): miR-409-3P, miR-886-5P, miR-324-3P, miR-222, miR-125A-5P, miR-339-3P, and/or miR-155.
